(19) United States
(12) Patent Application Publication
(10) Pub. No.: US 2022/0160296 A1
(43) Pub. Date: May 26, 2022

(54) PAIN ASSESSMENT METHOD AND APPARATUS FOR PATIENTS UNABLE TO SELF-REPORT PAIN

(71) Applicant: THE REGENTS OF THE UNIVERSITY OF CALIFORNIA, Oakland, CA (US)

(72) Inventors: Amir M. Rahmani, Irvine, CA (US); Nikil Dutt, Irvine, CA (US); Kai Zheng, Irvine, CA (US); Ariana Nelson, Orange, CA (US); Pasi Liljeberg, Turku (FI); Sanna Salantera, Turku (FI); Mingzhe Jiang, Turku (FI); Arman Anzanpour, Turku (FI); Elise Syrjala, Turku (FI); Riitta Mieronkoski, Turku (FI); Emad Kasaeyan Naeini, Irvine, CA (US); Ajan Subramanian, Irvine, CA (US); Seyed Amir HosseinAqajari, Irvine, CA (US); Rui Ca, Irvine, CA (US); Geng Yang, Hangzhou (CN)

(21) Appl. No.: 17/669,984

(22) Filed: Feb. 11, 2022

Related U.S. Application Data
(63) Continuation-in-part of application No. 16/406,739, filed on May 8, 2019, now abandoned.
(60) Provisional application No. 62/668,712, filed on May 8, 2018.

(51) Int. Cl. A61B 5/00; A61B 5/397; A61B 5/0205; A61B 5/024; A61B 5/0533; A61B 5/08; A61B 5/265

(52) U.S. Cl. CPC A61B 5/4824; A61B 5/397; A61B 5/02055; A61B 5/02427; A61B 5/0533; A61B 5/7264; A61B 5/0008; A61B 5/6803; A61B 5/7285; A61B 5/265; A61B 5/742; A61B 5/0816

(57) ABSTRACT

Systems and methods for automatic pain monitoring and assessment are described herein. In one example, the system may include a wearable facial expression capturing system that is placed over a subject's face. The system may be embedded with a plurality of sensors configured to detect biosignals from facial muscles and may additionally include a sensor node that recognizes facial expressions based on the detected biosignals. Pain experienced by the subject is assessed based on the facial expressions in conjunction with physiological signals obtained by other wearable sensors.

### CROSS-REFERENCES TO RELATED APPLICATIONS

This application is a continuation-in-part and claims benefit of U.S. Non-Provisional patent application Ser. No. 16/406,739, filed May 8, 2019, which claims benefit of U.S. Provisional Patent Application No. 62/668,712 filed May 8, 2018, the specification(s) of which are incorporated herein in their entirety by reference.

### GOVERNMENT SUPPORT

This invention was made with government support under Grant No./Funding Decision No. 286915 awarded by Academy of Finland. The government has certain rights in the invention.

### FIELD OF THE INVENTION

The present invention relates to systems and methods for pain assessment and continuous monitoring of pain in patients, more specifically in patients who are unable to report pain.

### BACKGROUND OF THE INVENTION

Pain is defined by the International Association for the Study of Pain (IASP) as “an unpleasant sensory and emotional experience associated with actual or potential tissue damage or described in terms of such damage”. Pain is a unique phenomenon that individuals experience and perceive independently. Pain may be classified as either acute or chronic; acute pain is described as encompassing the immediate, time-limited bodily response to a noxious stimulus that triggers actions to avoid or mitigate ongoing injury. Chronic pain was first defined loosely by Bonica as pain that extends beyond an expected timeframe; currently, chronic pain is defined as “persistent or recurrent pain lasting longer than three months”.

Acute pain is a common experience in the post-anesthesia care unit (PACU) in the immediate period following surgery. According to prior research, pain occurs in 80% of patients following surgery and 75% of patients with pain report their pain as either moderate, severe, or extreme.

Pain remains poorly managed partly because it is not recognized and assessed properly. Self-report is conventionally considered as the “gold standard”, which requires patients to answer questions verbally, in writing, with finger span or blinking eyes to yes or no questions. In the self-report method, the pain intensity is reported by the patient as numeric scales, which are based on two prerequisites: the patient's cognitive competence and unbiased communication. Current guidelines for the assessment of pain in the PACU recommend using a Numerical Rating Scale (NRS) or Verbal Rating Scale (VRS) for patients who are sufficiently awake and coherent to reliably report pain scores. Although taken as the “gold standard”, this unidimensional model is questioned and debated for its oversimplification and limitation in several vulnerable patient populations. However, there is no way to objectively assess patients' pain, especially from patients that have difficulties in communicating.

Several patient populations who are at risk for being incapable of providing self-report scores of pain have been identified; specifically, these populations include the pediatric population who have yet to develop adequate cognition, elderly patients with dementia, individuals with intellectual disabilities, and those who are unconscious, critically ill, or terminally ill. A broader range of non-self-report resources are observed by experienced clinicians to assess pain, for example, grimacing facial expression and body movements as behavioral observation and vital signs as physiologic monitoring. In these patient populations, the use of behavioral pain scales is recommended, such as the Pain Assessment in Advanced Dementia (PAINAD), Critical Care Pain Observation Tool (CPOT), or Behavioral Pain Scale (BPS). These non-self-report strategies are the theoretical basis and inspiration in developing an automatic pain assessment method to assist and even to replace the subjective self-report method.

Despite the pain assessment measures of self-report and behavioral pain scales, each of these methods may be prone to biases. For example, self-report may be a means to obtain a particular goal that can be influenced by the individual reporting pain. Additionally, the Communications Model of Pain provides a basis for how expressive behaviors are decoded by observers of individuals in pain, which are influenced by the message clarity transmitted by the individual in pain as well as the unique biases (e.g. knowledge level, assessment skills, and predisposing beliefs) of the individual assessing pain. The difficult nature of interpreting pain scores has resulted in disparities in pain management in minority populations, with research showing that the black race is a significant predictor of underestimation of pain by physicians.

In the past several decades, researchers and scientists have been trying to decode pain by monitoring electrical biosignals in different patient populations with a certain type of pain. So far, some correlation is found between electrical biosignals and pain but no individual one is sufficient enough to indicate the presence of pain due to the complexity of the automatic nervous system and pain expression. As a consequence, alternative comprehensive models of pain from multiple electrical biosignals are explored. Existing models built in the last five years mainly involve physiological pain indicators only from either healthy volunteers with a single type of experimental pain or patients in surgery and few have been applied in a different database for model validation. Furthermore, no model has yet been developed into an automatic pain assessment tool.

Multimodal pain assessment represents one potential method of circumventing the limitations of traditional self-report and behavioral pain assessment tools and an opportunity for enhancing pain assessment in vulnerable populations. Instead of having to rely on only one dimension of pain assessment, such as behaviors through the use of the CPOT or BPS scales, future multimodal pain assessment will incorporate physiological indicators, such as electrodermal activity (EDA), electrocardiogram (ECG), electroencephalogram (EEG) and electromyogram (EMG) as well as behaviors (e.g. facial expression), and perhaps other as-yet-undiscovered parameters to capture pain assessment in patient populations that might not be best represented by current assessment strategies. For example, prior studies have found that revisions to the CPOT were necessary because some brain-injured patients may not exhibit certain behaviors that are contained in the CPOT. Similarly, for individuals diagnosed with dementia, it has been stated that there is a preponderance of observer-based pain assessment tools, however, these tools retain significant differences between them, as well as concerns for lack of reliability, validity, and sensitivity of change. Enhancing pain assessment through the combination of traditional pain assessment methods with novel multimodal approaches may serve to eventually enhance pain assessment in a greater majority of vulnerable patient populations.

With the advent of connected Internet-of-Things (IoT) devices and wearable sensor technology, automated data collection may achieve continuous pain intensity measurement. A significant amount of research has been conducted in recent years which have sought to develop methods of continuous, automatic, and multimodal pain assessment. For example, prior work used skin conductance level (SCL), electrocardiogram (ECG), electroencephalogram (EEG), and electromyogram (EMG) to monitor pain in response to thermal pain. Other works have incorporated facial expression monitoring as an indicator of pain. While these studies were immensely beneficial to the scientific community in terms of their contributions to a better understanding of techniques to obtain continuous pain assessment, the setting of these experiments was in highly controlled laboratory environments from healthy participants. Collecting data in real-world situations as opposed to the laboratory provides two clear advantages: from a data collection perspective, conducting a study in a real-world environment provides an opportunity to assess interfering factors, such as noise from motion artifacts, baseline wander, and power channels; from a pain assessment perspective, this method would allow for the researchers to assess a pain assessment technique's potential in relation to actual pain brought about through a surgical procedure instead of an induced pain.

Prior systems have attempted to develop an efficient system for detecting pain intensity levels in a human patient. For example, “Method and Apparatus for Pain Management Using Objective Pain Measure” by Annoni, et al. teaches a system for managing pain of a patient through use of a pain monitoring circuit through a plurality of sensors on the body. “Electrode Assembly and Method for Signaling a Monitor” by Bennett, et al. teaches an electrode assembly adapted to be attached to the skin over selected facial muscle groups to pick up signals to be analyzed by an anesthesia adequacy monitor to measure the level of awareness of a living animal under anesthesia. “Mobile Wearable Electromagnetic Brain Activity Monitor” by Connor, et al. teaches a mobile wearable electromagnetic brain activity monitor for measuring electromagnetic brain activity while a person is ambulatory. “Multimodal Data Fusion for Person-Independent Continuous Estimation of Pain Intensity” by Kachele, et al. teaches a method for the continuous estimation of pain intensity based on the fusion of bio-physiological and video features. None of these prior references, however, implement a pain monitoring system that utilizes advanced multi-modal machine-learning methods such as early fusion and weak supervision to leverage complementary information available in different modalities. Furthermore, these prior art utilize a camera, whereas the presently claimed invention does not due to feasibility and privacy reasons.

The present invention features a method and a smart tool that assesses pain by utilizing physiological parameters monitored by wearable devices. Although pain is believed to be an individual sensation relying on subjective assessment, an objective assessment tool is needed for the wellbeing and improved care processes of non-communicative patients. Such a tool also benefits other patient populations with more accurate medication and clinical-assisted treatment.

### SUMMARY OF THE INVENTION

The present invention discloses a precise and automatic tool for pain assessment by biosignals acquisition and analysis with wearable sensor devices. Through monitoring behavioral and physiological signs, the appearance of pain and pain state is continuously tracked. The present invention additionally discloses the design of a wearable facial expression capture system and a data fusion method.

The present invention further provides automatic and continuous monitoring of pain intensity in patients who are otherwise unable to self-report. The real-time information of the continuous monitoring can be updated to a caregiver nearby or even in a remote location, so as to improve the nursing efficiency and optimize pain management in medication. The present invention includes a multi-modal integration of a plurality of physiological and behavioral signals to accurately estimate the pain experienced by the patient. Compared with a single monitoring of physiological signals or behavioral signals, a fusion or integration of the two potential pain indicators contributes to a more multidimensional and comprehensive model in automatic pain assessment. In addition, the integration of wearable devices ensures long-term monitoring in patients with lightweight and portable equipment.

This is the first work proposing a multimodal pain assessment framework for post-operative patients. It should be noted that a pain assessment study on real patients is associated with several challenges (e.g., imbalanced label distribution, missing data, motion artifacts, etc.) since several parameters such as the intensity, distribution, frequency, and time of the pain as well as the environment cannot be controlled by researchers. The main contributions are four-fold. A clinical study was conducted for multimodal signal acquisition from an acute pain unit of the University of California, Irvine Medical Center (UCIMC). The present invention features a multimodal pain assessment framework using the iHurt Pain database collected from post-operative patients while obtaining a higher accuracy compared to existing works on healthy subjects. The present invention uses both handcrafted and automatically generated features outputted from deep learning networks to build the models. The present invention features a novel method to mitigate the presence of sparse and imbalanced labels (due to the real clinical setting of the study) using weak supervision and minority oversampling.

Current pain assessment (PA) methods rely on caregivers asking patients to self-report their pain levels or observing behavioral or physiological pain responses and using context from the causes of pain. This assessment is often subjective in nature and is affected by social and personal factors including anxiety, depression, disability, and medication. Therefore, there is a pressing need to build an objective pain monitoring system that can predict pain intensity based on physiological factors.

The first step in designing such a system is to objectively measure behavioral and/or physiological responses to pain. Behavioral responses are used as protective mechanisms to bring attention to the source of pain and are communicated through facial expressions, body movements, and vocalizations. The use of facial expressions for pain assessment has been studied in-depth. Facial expressions are typically examined using the Facial Action Coding System (FACS), which breaks down expressions as movements of elementary Action Units (AUs) based on muscle activity. Facial expressions in response to pain are often varied and may co-occur with other emotions due to the subjective nature of pain experienced in a patient. Such responses can be measured using electromyogram activity (EMG). Physiological responses to pain stimuli are reflected in the autonomic nervous system's activities and can be measured through signals like electrocardiogram (ECG) or heart activity, electrodermal activity (EDA) or skin conductance, and respiratory rate (RR).

To build objective pain monitoring systems it is also very important to consider the type of subjects being recruited because the intensity of pain experienced is highly varied across different groups of people. Prior studies have focused on inducing pain on healthy subjects to reduce the impact of pre-existing conditions that might inject biases into the data (Biovid, BP4D, MIntPAIN, SenseEmotion, X-ITE Pain), whereas some studies have focused on patients with chronic pain (EmoPain, and UNBC McMaster database). In clinical settings, many patients suffer from ongoing chronic pain without the involvement of external stimuli, but pain response is oftentimes intensified through necessary medical procedures like surgeries. An underrepresented population in pain studies is patients suffering from acute postoperative pain. Prior works have focused on building pain assessment models on single modalities like ECG, EDA, and PPG from the postoperative pain study. Even though the results achieved for each of these single modalities are significant, prior systems have not leveraged the multimodal nature of the collected dataset. Building models using a single modality might not be able to capture the full extent of a patient's painful experience and often has caveats in some clinical contexts. Heterogeneous sources of data, on the contrary, could complement each other and lead to improved performance over any single modality. Therefore, building a multimodal pain assessment system that utilizes both physiological and behavioral responses to pain can prove to be vital for vulnerable patient populations.

In some aspects, the present invention features a facial expression capturing system for measuring pain levels experienced by a human. The system may comprise a flexible mask contoured to at least partially cover one side of the human's face, the mask having an eye recess or opening disposed between an elongated forehead portion of the mask, which is above the eye recess, and a cheek portion of the mask, which is beneath the eye recess; six sensor positions located on the mask such that two sensor positions are located laterally on the elongated forehead portion of the mask and the other four sensor positions located on the cheek portion of the mask and situated in a 2 by 2 arrangement; two or more sensors embedded in the mask, wherein each sensor occupies one of the sensor positions; a sensor node disposed on a lateral flap extending from the cheek portion of the mask, wherein the sensor node comprises a processing module and a transmitter; and connecting leads electrically coupling each of the two or more sensors to the sensor node. When the flexible mask is applied to partially cover one side of the human's face, the sensor positions align with pain-related facial muscles in the human's face, and the sensors are configured to detect biosignals from underlying facial muscles such as, for example, the frontalis, corrugator, orbicularis oculi, levator, zygomaticus, and risorius. In some embodiments, the processing module is configured to (i) receive the biosignals from the plurality of sensors, (ii) analyze the biosignals to deduce facial expressions and monitor pain intensity levels experienced by the subject based on the deduced facial expressions, and (iii) transmit the pain intensity levels to a medical care provider, thus allowing the medical care provider to continually monitor the pain intensity levels experienced by the subject thereby providing effective and efficient pain management.

In some embodiments, the flexible mask is composed of polydimethyl silicone elastomer (PDMS). In other embodiments, the sensors (104) comprise Ag/AgCl electrodes. The electrodes may be disposed on an inner surface of the mask (102) such that the electrodes are directly contacting skin when the mask is placed on the human's face.

In one embodiment, the system may include two sensors, where a first sensor occupies a distal-most sensor position located on the forehead portion of the mask, and a second sensor occupies a first row and first column of the 2 by 2 arrangement in the cheek portion of the mask. In a preferred embodiment, the first sensor can detect biosignals from a corrugator facial muscle and the second sensor can detect biosignals from a zygomatic facial muscle.

In another embodiment, the system may comprise five sensors, where a first sensor and a second sensor occupy the two sensor positions on the forehead portion of the mask, a third sensor and a fourth sensor occupy the sensor positions at a first row of the 2 by 2 arrangement in the cheek portion of the mask, and a fifth sensor occupies the sensor position at a second row and second column of the 2 by 2 arrangement. The first sensor can detect biosignals from a corrugator facial muscle, the second sensor can detect biosignals from a frontalis facial muscle, the third sensor can detect biosignals from a levator facial muscle, the fourth sensor can detect biosignals from an orbicularis oculi facial muscle, and the fifth sensor can detect biosignals from a zygomatic facial muscle.

In other aspects, the present invention provides a method for integrating surface electromyogram (sEMG) signals and physiological signals for automatically detecting pain intensity levels experienced by a human. One embodiment of the method may comprise providing a wearable facial expression capturing system for measuring said pain intensity levels. The system includes a flexible mask contoured to at least partially cover one side of the human's face, the mask having an eye recess or opening disposed between an elongated forehead portion of the mask, which is above the eye recess, and a cheek portion of the mask, which is beneath the eye recess; at least two sensors disposed in the mask, wherein a first sensor is disposed in the forehead portion of the mask, and a second sensor is disposed in the cheek portion of the mask; a sensor node disposed on a lateral flap extending from the cheek portion of the mask, the sensor node comprising a processing module and a transmitter; and connecting leads electrically coupling each of the at least two sensors to the sensor node. In some embodiments, more than two sensors may be implemented to add additional accuracy to the system but at a greater cost.

The method further comprises applying the flexible mask to partially cover one side of the human's face such that the first sensor aligns with a corrugator facial muscle and the second sensor aligns with a zygomatic facial muscle, detecting sEMG signals from the corrugator facial muscle and the zygomatic facial muscle via the first and second sensors, respectively, filtering the detected sEMG signals via the processing module, transmitting the filtered sEMG signals to a data fusion system via the wireless transmitter, and receiving physiological signals transmitted from one or more wearable sensors to the data fusion system. In some embodiments, the physiological signals may comprise one or more of a breath rate, a heart rate, a galvanic skin response (GSR), a skin temperature signal, or a photoplethysmogram (PPG) signal. The method continues with extracting features from each of the sEMG signals and the physiological signals, performing feature alignment on features extracted from the sEMG signals and the physiological signals, performing interindividual standardization on each of the sEMG signals and the physiological signals, performing pattern recognition by comparing the sEMG signals and the physiological signals to a database, correlating patterns recognized with pain intensity levels and classifying the pain intensity levels, and displaying the pain intensity levels to a medical care provider, thus allowing for continuous and automatic pain monitoring.

In one embodiment, the step of extracting features from each of the sEMG signals and the physiological signals may comprise a root-mean-square (RMS) feature extraction and a wavelength (WL) feature extraction. In another embodiment, the step of performing feature alignment includes synchronizing the sEMG signals and the physiological signals by using cross-correlation functions. In an additional embodiment, the step of correlating patterns recognized with pain intensity levels and classifying the pain intensity levels are performed using an artificial neural network classifier.

The present invention implements a combination of feature alignment and early fusion on features extracted from the sEMG signals and the physiological signals. Early fusion is referred to as input level fusion. One early fusion approach (see FIG. 1) is to fuse data at its lower-dimensional common space. Early fusion is applicable to raw data or pre-processed data obtained from sensors. Data features should be extracted from the data before fusion, otherwise, the process will be challenging especially when the data sources have different sampling rates between the modalities. Hence, converting data sources into a single feature vector is a challenge in early data fusion. Despite this challenge, the present invention is able to implement early fusion for integrating information from multiple modalities to predict an outcome measure, mainly, pain monitoring and management. The implementation of early fusion allows the present invention to leverage advanced multimodal integration of a plurality of physiological and behavioral signals to accurately estimate the pain experienced by a patient.

One of the unique and inventive technical features of the present invention includes the wearable mask for facial expression capture and for pain assessment, pain management, and clinical monitoring. Without wishing to limit the invention to any theory or mechanism, it is believed that the technical feature of the present invention advantageously provides for aligning embedded sensors on the mask with facial muscles that are activated when experiencing pain, thereby maximizing the signals detected by the sensors, and further enhancing the sensitivity of the system for measuring pain experienced by the patient.

Another unique and inventive technical feature of the present invention includes analyzing a plurality of physiological signals and comparing the signals with one another and/or a database to correlate the measured physiological signals with pain intensity values. In this way, an accurate measure of the pain levels experienced by the patients may be determined. By continuously monitoring the pain levels and displaying the detected pain levels, a medical provider may be able to make intelligent and effective pain management decisions for the patient, thereby improving quality of life in patients suffering from constant or complex pain, for example.

In addition, the current belief in the prior arts is that the incorporation of the sensors into a mask would interfere with detection. Although the sensors were localized, it was thought that the mask would couple the sensors together such that movement of one sensor would affect other sensors, resulting in noise and inaccurate signal detection. It was also thought that the mask would add significant weight to dislocate the sensors from the desired positions on the human face. Thus the prior art teaches away from the present invention. However, contrary to prior teachings, the embedding of the sensors into the mask of the present invention surprisingly worked and was able to detect signals related to pain expression from the individual facial muscles without exhibiting signal or placement issues. Furthermore, the multimodality resulting from the integration of surface electromyogram (sEMG) signals obtained by the wearable sensor mask and other physiological signals obtained by other sensor devices produced a synergistic effect that enhanced detection of the pain responses and distinguished them from other biological responses in the human. As such, none of the known prior references or work has the unique inventive technical features of the present invention.

Another unique and inventive technical feature of the present invention is the assessment of pain in a patient without the use of any camera. This provides great advantages to user privacy as no images of the said user's face need to be captured and/or stored by the computing device of the present invention. Furthermore, the obviation of a camera increases the cost efficiency and comfort of patients, and the implementation of EMG signals instead of camera images greatly increases the accuracy of the invention and allows for the measurement of smaller micromovements of the face. None of the known prior references or work has the unique inventive technical features of the present invention.

Furthermore, the unique technical feature of the present invention contributed to a surprising result. One skilled in the art would expect that the collection of signals without any images gathered by a camera would be unable to accurately measure pain in a patient based on facial movements since signals could potentially inaccurately identify a reaction to pain where there was none (e.g. facial expressions due to emotion). Surprisingly, the present invention is able to more accurately identify pain in a patient through the use of a plurality of signals (including facial EMG which captures micro facial muscle movements) without needing to implement images by a camera. Thus, the unique technical feature of the present invention contributed to a surprising result.

Another unique and inventive technical feature of the present invention is the implementation of a weak supervision algorithm executed on the raw data collected from the sensors of the multi-modal system for pain monitoring purposes. Without wishing to limit the invention to any theory or mechanism, it is believed that the technical feature of the present invention advantageously provides for the ability to efficiently label a large amount of data collected from the patient to prep said data for feature extraction. None of the known prior references or work has the unique inventive technical features of the present invention.

Furthermore, the inventive technical feature of the present invention teaches away from the prior references. Prior works have only tried their solution on health subjects who are able to self-report their pain score regularly (used as labels in the supervised machine learning), but when their models are used in the field, their accuracy drops. The present invention implements collected data in realistic settings where the subject is unable to self-report their pain score regularly and cope with the issue of irregular and scare labels (self-reported scores) by using weak supervision and minority sampling.

Furthermore, the inventive technical feature of the present invention contributed to a surprising result. One skilled in the art would implement supervised learning with hand-labelled data to maximize the accuracy of the system for pain detection. Surprisingly, the weak supervision method (getting help from machine/AI to label some extra datapoints for us instead of humans) worked well and increased the prediction accuracy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1A shows a wearable facial expression capturing system placed on a subject's face, where the system comprises a facial mask embedded with a plurality of electrodes, according to an embodiment of the present invention.

FIG. 1B is a non-limiting example of a prototype of the facial mask embedded with a plurality of electrodes.

FIG. 1C shows the prototype placed on a subject's face.

FIG. 1D shows another non-limiting example of a wearable facial expression capturing system having a face mask with two electrodes.

FIG. 1E is another non-limiting example of a wearable facial expression capturing system having a face mask with five electrodes.

FIG. 2 shows a non-limiting example of a pain assessment system that continuously monitors pain from subjects and reports it to a data fusion system which displays the data to a medical care provider.

FIG. 3 shows a non-limiting example of a pain assessment system that continuously monitors pain from subjects and reports it to a cloud and a remote database, which is then accessed by the medical care provider.

FIG. 4 shows a high-level flow chart depicting an example method for detecting biosignals from the plurality of electrodes and physiological signals and analyzing the signals to recognize facial expressions and deduce pain levels based on the facial expressions.

FIG. 5 shows an example plot showing surface electromyogram (sEMG) signals from eight channels of the system.

FIG. 6 shows an example scatter plot of the RMS features of four expressions from one fold training dataset with three of the four channels of sEMG signals.

FIG. 7 shows a schematic diagram of a pain stimulation and biosignal measurement environment.

FIG. 8 shows a timeline of a test for measuring pain intensity levels.

FIG. 9 shows a schematic diagram of a data processing flow and classification of matrices.

FIG. 10 shows Pearson's linear correlation coefficient between pain intensity levels and parameters in the matrices. The physiological parameters on the horizontal axis are sorted in descending order of coefficient of absolute value.

FIGS. 11A and 11B show the distribution of the area under the curve (AUC) from classification with a different number of sEMG parameters in addition to heart rate, breathe rate, and galvanic skin response.

FIG. 12 shows a schematic of biosignal acquisition using the system of the present invention. The signals collected were electrocardiogram (ECG), electromyogram (EMG), electrodermal activity (EDA), and photoplethysmogram (PPG).

FIG. 13 shows a table of extracted respiratory rate (RR) features and their descriptions.

FIG. 14 shows a flow chart of the handcrafted feature extraction pipeline of the present invention.

FIG. 15 shows an architecture of the pyEDA convolutional autoencoder used in the present invention.

FIG. 16 shows a flow chart of the automatic feature extraction pipeline of the present invention.

FIG. 17 shows a proposed general multimodal pipeline based on early fusion (left) and late fusion (right).

FIG. 18A shows a table of single modality scores from experimentation.

FIG. 18B shows a table of multiple modality scores from experimentation.

FIG. 19 shows the best classifier configurations for each pain intensity.

### DESCRIPTION OF PREFERRED EMBODIMENTS

Following is a list of elements corresponding to a particular element referred to herein:
100 wearable facial expression capturing system
102 mask
104 electrode/sensor
106 connecting lead
108 sensor node
112 wireless transmitter
114 subject's face
115 eye recess or opening
116 forehead portion of mask
117 cheek portion of mask
118 lateral flap
200 pain assessment system
202 pain detection system
204 monitoring system
206, 212 patient
208, 214 wearable facial expression capturing system
210, 216 wireless transmitter
218 gateway
220 display
222 medical care provider
226 cloud
228 remote server
300 pain monitoring system
302 wearable facial expression capturing system
304 electrode/sensor
306 sensor node
308 wireless transmitter
310 processing module
312 wearable sensors
314 ECG sensor
316 breath rate sensor
318 heart rate sensor
320 PPG sensor
322 data fusion system
324 WIFI receiver
326 memory
328 processor
330 display

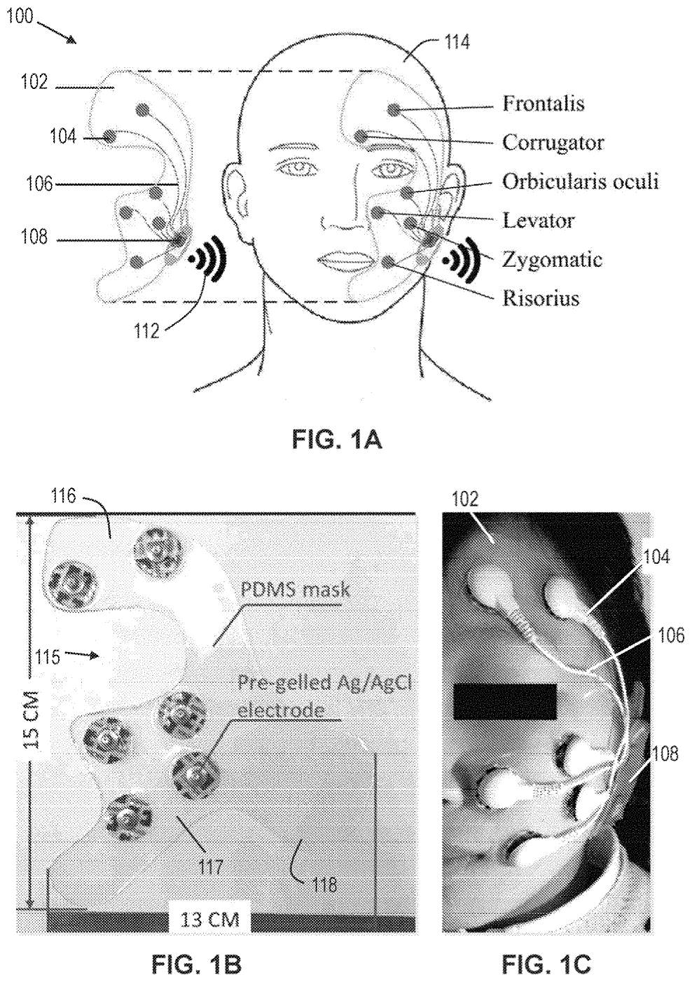

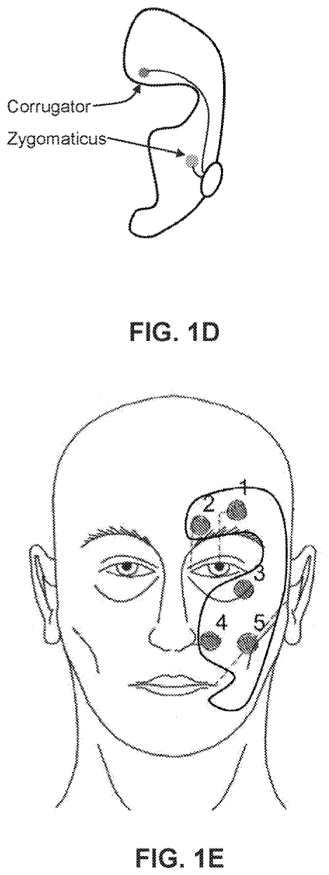

Referring now to FIG. 1A-11B, the present invention features a real-time pain monitoring system for subjects, who are unable to self-report, for example, to improve the efficiency of reporting and optimizing pain management and medication. The present invention discloses a wearable facial expression capturing system (100) positioned over a subject, as shown in FIGS. 1A-1E. In some embodiments, the system (100) comprises a mask (102) made of a soft and pliable material, which can conform to the shape of the subject's face (114). As a non-limiting example, the mask (102) may be composed of poly dimethyl silicone elastomer (PDMS) substrate which is soft, stretchable, transparent, and lightweight, and which can be worn on the face. As such, the softness of PDMS makes the mask fit well on the curvature of the user's face. Other materials may be used for creating the mask without deviating from the scope of the invention.

In some embodiments, a thickness of the mask (102) may be selected based on one or more of desired flexibility and overall weight for user comfort, for example. As a non-limiting example, the thickness of the mask (102) may be about 50-150 μm. In one non-limiting example, the thickness of the manufactured mask may be about 100 μm. As a non-limiting example, the overall weight of the mask may be about 7-10 g. In one non-limiting example, the weight of the mask may be about 7.81 g. Other values of thickness and weight may be used without deviating from the scope of the invention.

In some embodiments, the mask (102) is implemented by integrating detecting electrodes into the soft polydimethylsiloxane (PDMS) substrate. As a result, the designed mask is easy-to-apply, and offers a one-step solution, which can largely save the valuable time of the caregivers when making setting up for sensing vital bio-signals from patients, in particular in the ICU ward environment. In a non-limiting embodiment, the mask (102) is integrated with a plurality of sensors or electrodes (104) embedded into the mask (102), such that when worn, the plurality of electrodes (104) are in contact with specific detection points on the subject's face (114). In one non-limiting example, the plurality of sensors (104) may include electrodes for detecting surface electromyogram (sEMG) signals from facial muscles. As an example, the electrodes may include six pre-gelled Ag/AgCl electrodes positioned at specific locations (positions 1-6 shown in FIG. 1A-1C) on the mask. In terms of pain-related facial expressions, the main facial muscles that are involved are listed in Table I.

TABLE 1
Pain related facial muscles and the targeted facial action units (AU).

| Channel | Muscular basis | AU |
|---|---|---|
| 1 | Frontalis | |
| 2 | Corrugator | Brow lower (AU 4) |
| 3 | Orbicularis oculi | Lids tighten (AU 6) |
| | | Cheek raise (AU 7) |
| 4 | Levator | Nose wrinkle (AU 9) |
| | | Upper lip raiser (AU 10) |
| | | Eyes close (AU 43) |
| 5 | Zygomatic | Lip corner pull (AU 12) |
| 6 | Risorius | Horizontal mouth stretch (AU 20) |

In some embodiments, fewer electrodes may be used to detect biosignals from the facial muscles to recognize facial expressions. As a non-limiting example, four electrodes may be positioned to line up with the corrugator, orbicularis oculi, levator, and the zygomatic to study the facial expressions. In other embodiments, as shown in FIG. 1D, the facial mask may include two sensors positioned to line up with the corrugator and the zygomatic. In another non-limiting example, as shown in FIG. 1C, the facial mask may include five sensors positioned to line up with corrugator, frontalis, orbicularis oculi, levator, and the zygomatic. In alternative embodiments, additional reference electrodes may be included in the mask. The reference electrode may be positioned on the bony area behind the ear, for example.

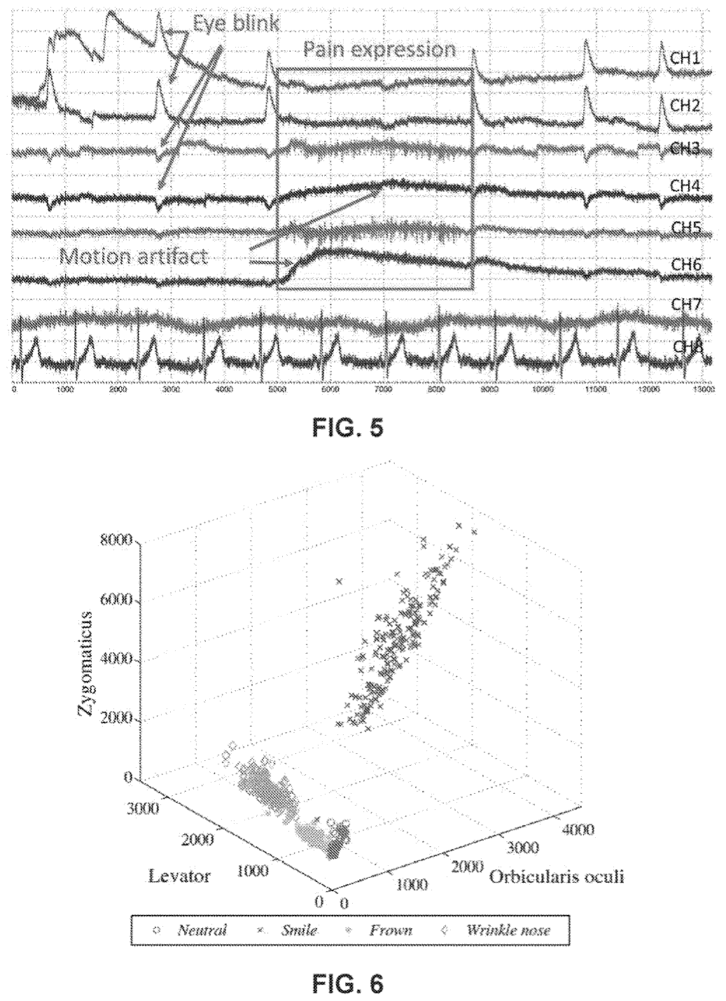

To recognize facial expressions with sEMG method, three to eight channels of sEMG signals may be used. An example plot showing sEMG signals from eight channels is shown in FIG. 5. The sEMG signals may be analyzed to ascertain the facial expression, as described further below.

Each electrode (104) is aligned with the facial muscles of table 1. Herein, a spacing between individual electrodes is selected such that each electrode overlays on top of a muscle from table 1. Each electrode (104) is integrated on the inner side surface of the mask (102) and closely attached to facial skin for reliable surface electromyogram (sEMG) measurement. These are passive electrodes and can be as small as 1 cm×1 cm or smaller. The placement of the electrodes is determined by the targeted facial muscles. Due to the soft nature of the implemented mask, the electrode position and the shape of the mask can be slightly adjusted accordingly to accommodate individual facial differences. The plurality of electrodes may be printed and personalized to a subject's facial muscles to maximize accuracy.

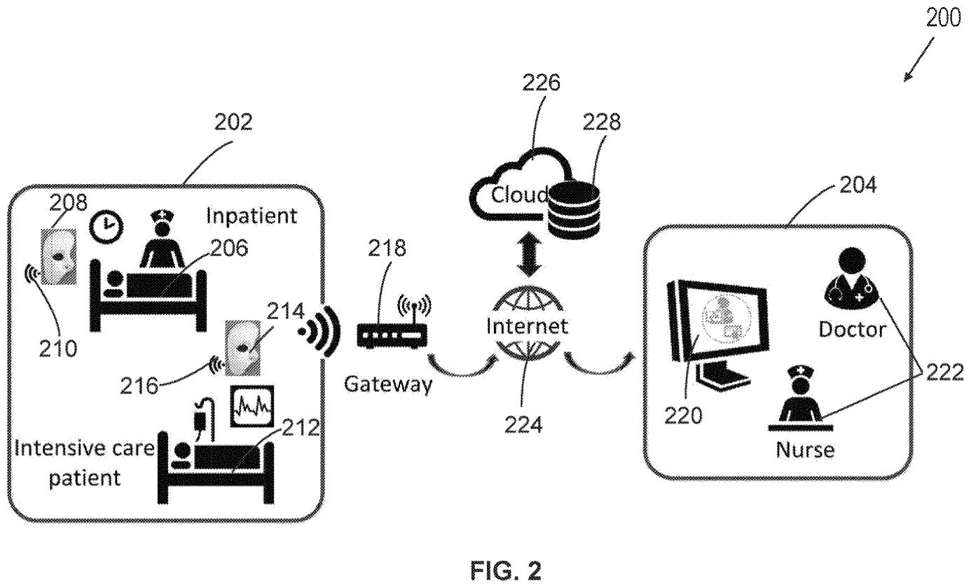

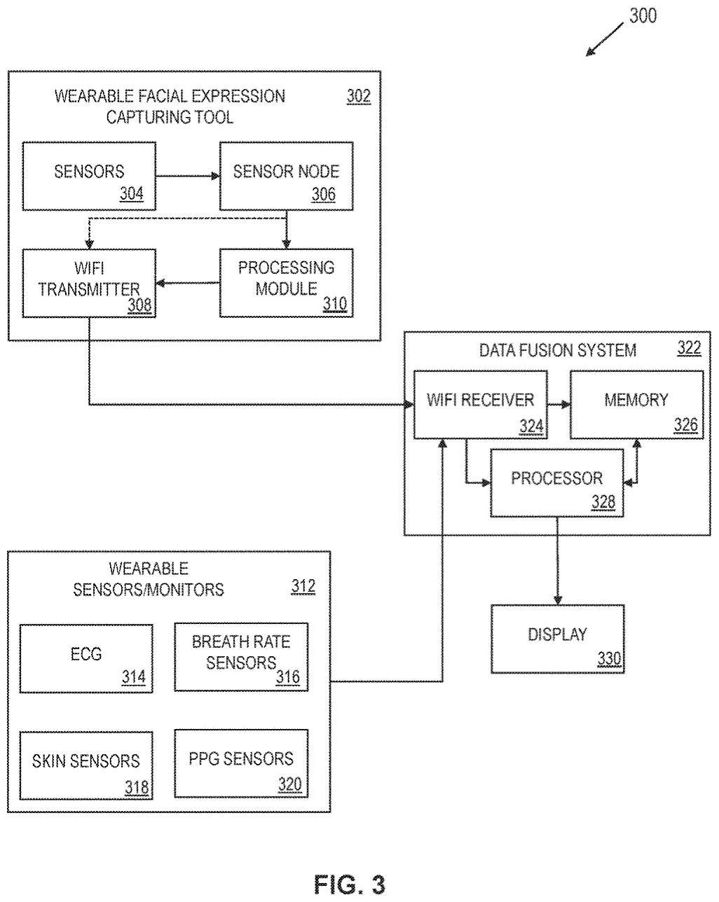

Each electrode (104) is electrically coupled to a sensor node (108) via connecting leads (106). As an example, the connecting leads (106) may be snapped or clipped on to the electrode (104) embedded on the mask (102). Herein, the connecting leads may be positioned along a top surface of the mask (102). The sensor node (106) may receive biosignals or sEMG signals detected by the electrodes via the connecting leads (106). The sensor node (108) may include a processing module that is configured for conditioning and digitizing the biosignals. The sensor node (108) may additionally include a wireless transmitter (112) that is configured to wirelessly transmit the biosignals to a receiver end, as shown in FIGS. 2 and 3. In one non-limiting example, the sensor node (108) may be attached behind the ear. In other examples, the sensor node (108) may be positioned on the neck. As such, the sensor node (106) may be positioned at other locations without deviating from the scope of the invention.

Turning now to FIG. 2, a schematic diagram of an example pain assessment system (200) that continuously monitors pain from several subjects and reports the data to a medical care provider (222) for pain management is shown. The system (200) comprises a signal detection system (202) that detects biosignals from multiple patients each wearing a wearable facial expression capturing system (208, 214). The wearable facial expression capturing systems (208, 214) may be non-limiting examples of the wearable facial expression capturing system (100) shown in FIG. 1. For example, the detection system (202) may detect biosignals from a first patient (206) wearing the wearable facial expression capturing system (208) and may transmit the biosignals of the first patient (206) wireless through a wireless transmitter (210) of the system (208) to a cloud (226) or remote server (228) wirelessly via a gateway (218). The detection system (202) may additionally detect biosignals from a second patient (212) wearing the wearable facial expression capturing system (214) and may transmit the biosignals of the second patient (212) to the cloud (226) or remote server (228) wirelessly via the gateway (218). In the cloud (226) or server (228), signals from the signal detection system (202) may be processed and classified after which it is sent to a monitoring system (204) where the signals are displayed to a medical care personnel (220) such as a nurse or doctor via a display (220). Herein, the display may include any device that is capable of visually displaying the signals such as a monitor, mobile phone, laptop, table, for example. Processing of the biosignals may include filtering, segmenting, and performing feature extraction, as described below.

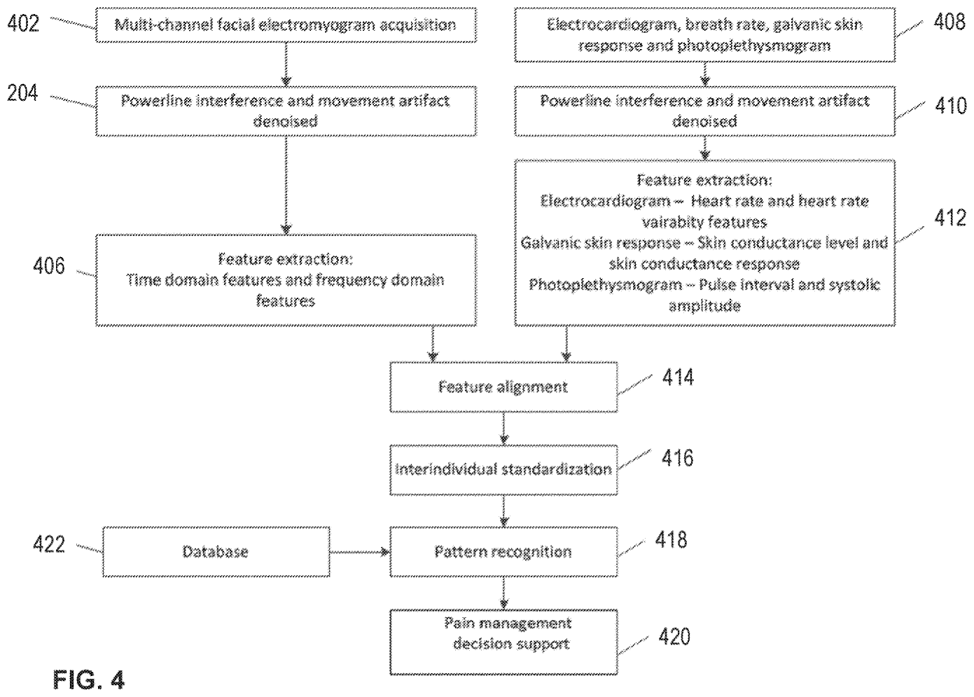

Current acute pain intensity assessment tools are mainly based on self-reporting by patients, which is impractical for non-communicative, sedated or critically ill patients. The present invention discloses continuous pain monitoring systems and methods with the classification of multiple physiological parameters, as shown in FIGS. 3 and 4. Turning now to FIG. 3, a schematic diagram of a pain monitoring system (300) is shown. The pain monitoring system (300) may include a wearable facial expression capturing system (302), additional wearable sensors (312), and a data fusion system (322). The wearable facial expression capturing system (302) may be a non-limiting example of the wearable facial expression capturing system (100) described in FIG. 1. As described previously, the wearable facial expression capturing system (302) may include a mask that is placed on a subject's face. The mask may include a plurality of embedded sensors (304), a sensor node (306), a processing module (310), and a WIFI transmitter (308). As explained previously, the system (302) may detect biosignals or biopotentials or sEMG from the surface of the face. Herein, the sEMG signal is a voltage produced by the facial muscles, particularly muscle tissue during a contraction.

In some embodiments, facial sEMG signals may be gathered when the person is with a neutral expression and facial expressions such, smile, frown, wrinkle nose, and the like. The sEMG signals detected by the plurality of sensors (304) may be sampled as different channels. As an example, when four electrodes are placed on the muscles to detect sEMG signals, four channels may be sampled at 1000 SPS. After the sampling, the signals may be filtered. In a non-limiting example, the sampled signals may be filtered using a 20 Hz high pass Butterworth filter and a 50 Hz notch Butterworth filter. As such, the filtering of the signals reduces the artifacts and power line interference coupled to the connecting leads. The sEMG signals may be segmented into 200 ms slices, for example. In some embodiments, the sEMG signals may be filtered by the processing module (310). In some embodiments, the sEMG signals may be transmitted to a removed server/cloud, as shown in FIG. 3, where the signals are analyzed. In some embodiments, the sEMG signals may be transmitted to a data fusion system (322) for further analysis. The data fusion system (322) may include a WIFI receiver (324) configured to receive the sEMG signals from one or more systems (302) and the remote server/cloud. The data fusion system (322) may additionally include a memory (326) and a processor (328) for storing and performing processing steps, as disclosed below.

In some embodiments, the data fusion system (322) may receive raw sEMG signals from the system (302), and the processor (328) may filter and segment the sEMG signals. In some embodiments, the data fusion system (322) may receive filtered and segmented sEMG signals.

Once the sEMG signals and filtered and segmented, a root-mean-square (RMS) feature extraction may be performed on the signals. Mathematically, the RMS features are extracted using the following equation:

$$RMS = \sqrt{\frac{1}{N} {\sum }_{i = 1}^{N} {x}_{i}^{2}} \qquad ( 1 )$$

The RMS feature extraction may provide insight on sEMG amplitude in order to provide a measure of signal power, for example. Wavelength (WL) feature extraction may be additionally or alternatively performed on the sEMG signals as a measure of signal complexity. WL features are extracted using the following equation:

$$WL = {\sum }_{i = 1}^{N - 1} \vert {x}_{i + 1} - {x}_{i} \vert \qquad ( 2 )$$

A multivariate classifier is trained for expression classification. Parameters of Gaussian distribution for each expression are estimated from training data, i.e. a feature matrix. Herein, the feature matrix may include signals for neutral, smile, frown, wrinkle nose, and the like. In some embodiments, the feature matrix may be stored in the memory (326) of the data fusion system (322).

Then the posterior probability of a given class c in the test data is calculated for pattern recognition. The equation below is Bayes theorem for the univariate Gaussian, where the probability density function of continuous random variable x given class c is represented as a Gaussian with mean μ_{c} and variance σ_{c}2.

$$P ( x ) ∝ \frac{1}{2 {πσ}_{c}} exp ( \frac{- {( x - {μ}_{c} )}^{2}}{2 {σ}_{c}^{2}} ) P ( c ) \qquad ( 3 )$$

In this way, the sEMG signals may be compared with the feature matrix, and the facial expression may be recognized based on the comparison. As an example, when employing multivariate Gaussian classifier, 10 fold cross-validation is applied and the classification accuracy is about 82.4%. The scatter plot of the RMS features of four expressions from one fold training dataset with three of the four channels sEMG are shown in FIG. 6. Each test dataset is combined by four expressions in the sequence of neutral, smile, frown and wrinkle nose.

In addition to extracting facial expressions based on sEMG signals detected from the facial expression capturing system (302), the pain monitoring system (300) may be configured to receive signals from other wearable sensors/monitors (312). Some non-limiting example of the wearable sensors/monitors include heart rate (HR) sensors, breath rate (BR) sensors, galvanic skin sensors, photoplethysmogram (PPG) sensors, and the like. As an example, the wearable sensor (312) may be a watch that is worn on the wrist and monitors the heart rate. As another example, the wearable sensor (312) may be a monitor that is worn on a chest and torso for monitoring the heart rate. As yet another example, the wearable sensor may be the PPG sensor worn on a finger to monitor pulse oxygen in the blood. Other examples of wearable sensor include biopatches and electrodes worn/attached to anywhere on the body.

The pain monitoring system may receive signals from the wearable sensor (312). Herein, the signals received may include one or more of a heart rate (HR), a breath rate (BR), a galvanic skin response (GSR), a PPG signal, and the like. The processor (328) may filter the signals received from one or more of the wearable sensors (312) to remove powerline interference, baseline wander, and movement artifacts. The processor (312) may additionally perform feature extraction on the signals received from the wearable sensors. Some examples of feature extraction may include extracting heart rate and heart rate variability features from the ECG, extracting skin conductance level and skin conductance response from the skin sensors, and extracting pulse interval and systolic amplitude from the PPG signal. Other features may be extracted without deviating from the scope of the invention. The processor may combine the sEMG feature extraction and the sensor feature extraction to monitor and manage pain, as described in FIG. 4.

Turning to FIG. 4, an example method (400) for integrating sEMG signals with additional biosignals to monitor pain levels in subjects is shown. Instructions for carrying out method 400 included herein may be executed by a processor based on instructions stored on a memory of the processor and in conjunction with signals received from sensors of the pain monitoring system, such as the sensors described above with reference to FIGS. 1A-3. Method 400 includes acquiring multi-channel facial sEMG signals at 402. As described previously, the sEMG signals may be detected using a wearable facial expression capturing system such as the system (100) described in FIGS. 1A-1E. At 404, method 400 includes powerline interference and movement artifact denoising, and method (400) proceeds to 406, where the facial features are extracted, as described in detail with reference to FIG. 3. As an example, denoising may include removing powerline interference and movement artifact from the signals. For most of the raw biopotential data, contamination by the environmental noise or human body movement is inevitable. One common contamination source among biopotential signals is power line interference, composed of 50 Hz or 60 Hz and its harmonics. Another common noise source in EMG is body movement that dominates low-frequency part of the signal. Therefore, denoising is the basic processing applied in biopotential signals. A variety of filters from FIR or IIR, adaptive ones, to wavelet method can be applied in terms of noise cancellation in order to improve the signal to noise rate.

At 406, the feature extraction may include extracting time domain and frequency domain features of the sEMG signals using RMS and WL features, as described in equations (1) and (2).

Method 400 may simultaneously receive and process physiological signals from other wearable devices as described with reference to FIG. 3. For example, at 408, method 400 includes receiving one or more of HR, BR, GSR, and PPG signals from wearable devices (such as devices (312 shown in FIG. 3). Like step 404, at 410, method 400 includes denoising the signals received at 408. Next, at 412, method 400 includes extracting features from the signals of the wearable sensors. Some examples of the feature extraction may include extracting heart rate and heart rate variability features from the ECG, extracting skin conductance level and skin conductance response from the skin sensors, and extracting pulse interval and systolic amplitude from the PPG signal. In some embodiments, RMS and/or WL feature extraction (equations (1) and (2)) may be performed on the signals from the wearable sensors to extract the features.

At 414, method 400 includes performing time alignment on the features extracted from sEMG signals, and from the signals such as HR, BR, GSR, PPG, and the like. As such, the sEMG, HR, BT, GSR, and PPG measurements may include signals collected asynchronously by multiple sensors. In order to integrate the signals and study them in tandem, the signals have to be synchronized. In one non-limiting example, the sEMG signals may be aligned with the HR, BR, GSR, PPG signals using cross-correlation functions. Other techniques may be used to synchronize the signals, without deviating from the scope of the invention.

At 416, method 400 includes performing interindividual standardization or normalization. The interindividual standardization includes rescaling the range and distribution of each signal. Rescaling may be used to standardize the range of the sEMG signals and the physiological signals. As such, the standardization of the signals may reduce subject-to-subject and trial to trial variability. In one embodiment, the signals may be standardized by equation (4) shown below:

$$Z = \frac{( X - μ )}{σ} \qquad ( 4 )$$

where X is the feature, μ is the mean, and σ is the standard deviation. The standardization results in generating a parameter matrix. As an example, the standardization of the sEMG signals may result in a matrix containing one set of RMS features and another set of WL features. For example, for sEMG signals arising from five face muscles, the parameter matrix may include ten standardized values. In addition, the parameter matrix includes standardized physiological signals such as HR, BR, and GSR. Thus, the standardization of the sEMG signals and the physiological signals may generate a 13-dimensional parametric matrix.

At 418, method 400 includes performing pattern recognition. The sEMG signals and the BR, HR, and GSR, signals may be compared with corresponding feature matrices stored in the database (422). Based on the comparison, method 400 may classify the signals into no pain, mild pain, or moderate/severe pain. Herein, the parameters of a built model may be trained by the existing database. The model may then be used to classify the new coming features. The model may also be later updated by retraining with the updated database which involves the labeled new coming features. In one embodiment, the comparison may include performing correlation analysis between the physiological parameters, sEMG, and pain intensity levels. As an example, GSR, HR, and BR in the parameter matrix may be used as predicting. Herein, GSR and HR positively correlated with pain intensity level, indicating that these two parameters were more likely to increase when a healthy subject experiences a high intensity of pain, while BR decreases. Among facial sEMG parameters, ZygRMS includes a greater correlation to the pain intensity level than others. GSR, HR, BR and two corrugator superclii parameters in the median matrix showed a stronger correlation to the pain intensity level than the parameter matrix. As such, the medians of both corrugator supercilii parameters showed considerable potential for differentiating pain intensity levels. Thus, transient responses of facial expressions may correlate to acute pain. In some embodiments, Pearson's linear correlation analysis may be used to compare the sEMG signals and physiological signals with pain intensity levels.

Thus, the present invention discloses automatic pain monitoring by classification of multiple physiological parameters. In addition, by performing parameter matrix classification where the physiological parameter samples are classified every second, it may be possible to continuously monitor pain. The physiological parameters are either clinically accessible or available from wearable devices and are appropriate for continuous and long-term monitoring. Besides, this monitoring method may help clinicians and personnel working with patients unable to communicate verbally to detect their acute pain and hence treat it more efficiently.

Examples of Medical Use Cases: Post-Operative Pain Assessment and Patient Behavior Assessment (e.g., Blink and Swallowing)

The automatic pain detecting system and method disclosed herein may be used to detect pain in non-communicative subjects. As an example, in emergency rooms or in ambulances, where patients are sometimes unable to communicate, the present invention may be used to automatically detect the level of pain that the patient is experiencing. As another example, for premature babies or infants or people with cognitive disabilities such as Alzheimer's or dementia, the present invention may be used to automatically detect the level of pain experienced by the subject. Once the pain levels are determined, the medical care provider may be able to administer the proper treatment or prescribe the correct levels of pain medications, for example.

In some situations, the medical provider may need to assess if the pain is real. For example, in subjects who are opioid/substance users, the medical provider cannot rely on the communication from the subjects. There needs to be an independent and more accurate measure of pain levels, so that the medical provider may be able to corroborate the results with the verbal communication received from the subjects. In this way, the medical provider may be able to selectively prescribe pain medications only when the pain is real.

The present invention may be used in situations to regulate the pain medication dosage. As an example, in postoperative patients who need persistent pain prevention, the present invention may be used to automatically detect the pain levels, thereby providing the medical care provider with an accurate measure of the pain levels experienced by the patients, so that the provider can adjust the dosage of the pain medications based on the measured pain levels. In some examples, the present invention may be used to assess pain in palliative or home care patients. In some more examples, the present invention may be used for the detection/prevention of breakthrough pain in cancer. The present invention may also be used to detect work-related stress and other unhealthy distress experienced by subjects.

### Example 1

The following is a non-limiting example of the present invention. It is to be understood that said example is not intended to limit the present invention in any way. Equivalents or substitutes are within the scope of the present invention.

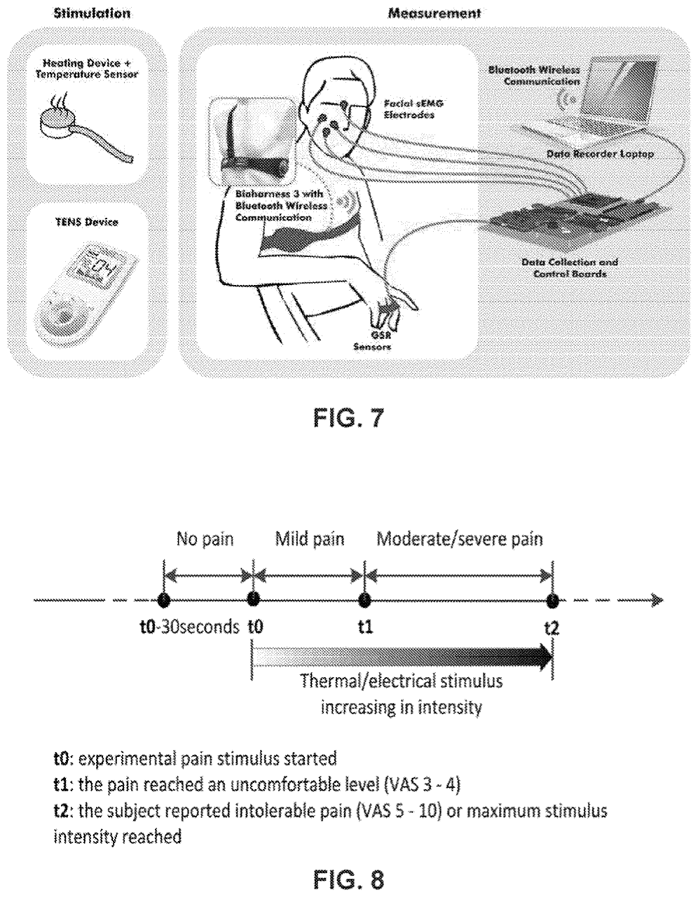

To develop a continuous pain monitoring method from multiple physiological parameters with machine learning, HR, BR, GSR, and facial surface electromyogram (sEMG) were monitored from healthy volunteers under experimental pain stimulus (FIG. 7). Facial expressions were captured from sEMG of the skin above five pain expression-related facial muscles: corrugator supercilii, orbicularis oculi, levator labii superiors, zygomaticus major, and risorius. Two types of experimental pain stimuli, thermal stimuli (heat) and electrical stimuli, were employed on both the right and left sides of the body in the study to cover more than one dimension of pain perceptions. Three pain intensity levels—no pain, mild pain, and moderate/severe pain—were collected from self-reports with visual analog scale (VAS) and were defined as three categories in classification (shown in FIG. 8).

### Biopotential Measurement

Physiological signals including HR, BR, GSR, and five facial sEMG from the right side of the face were continuously recorded throughout the session. FIG. 7 shows a brief description of the measurement environment, where GSR was captured from pre-gelled Ag/AgCl electrodes on the finger, five channels sEMG were captured from the Ag/AgCl electrodes on corrugator supercilii, orbicularis oculi, levator labii superiors, zygomaticus major, and risorius on the face, and HR and BR were from a Bioharness® belt worn on the chest. HR, BR, and GSR were taken at one second time resolution and sEMG were sampled with a Texas Instruments 8 channel biopotential measurement device at a rate of 1000 samples per second.

### Study Design

The study subject was seated in an armchair. At the beginning of the study session, the sensors and the device were established and it was ensured that they were able to record and appropriately catch the signals from all devices. The pain was induced by thermal and electrical stimuli in a random fashion, two times for each stimuli. The subjects were tested four times during each session and the tests were 1) electrical stimuli on the right-hand ring finger, 2) electrical stimuli on the left-hand ring finger, 3) thermal stimuli on the right inner forearm, and 4) thermal stimuli on the left inner forearm. The pain exposure starting location was randomized and the change of stimulated skin site helped in avoiding habituation to repeated experimental pain. Each data collection session started by letting the subject settle down and rest for ten minutes, so as to acquaint himself or herself with the study environment. Pain testing was only repeated after the subject's HR and BR had returned (if changed) to their respective baseline level.

The intensity of pain was evaluated using VAS at two time points: t1—when the pain reached an uncomfortable level (VAS 3-4), and t2—when the study subject reported intolerable pain or when stimulus intensity reached the non-harmful maximum. The time points and data definition are illustrated in FIG. 8. To balance the data size of each class, data of the 30 seconds before applying pain stimulus was labeled as no pain. During pain stimulation, data from when it started to when it reached an uncomfortable level was labeled as mild pain. The second part of the data under pain stimulus was marked as moderate/severe pain, where either moderate or severe depends on the VAS the study subject reported. All physiological signals were marked with time stamps and were saved for offline processing along with VAS evaluations.

### Data Pre-Processing

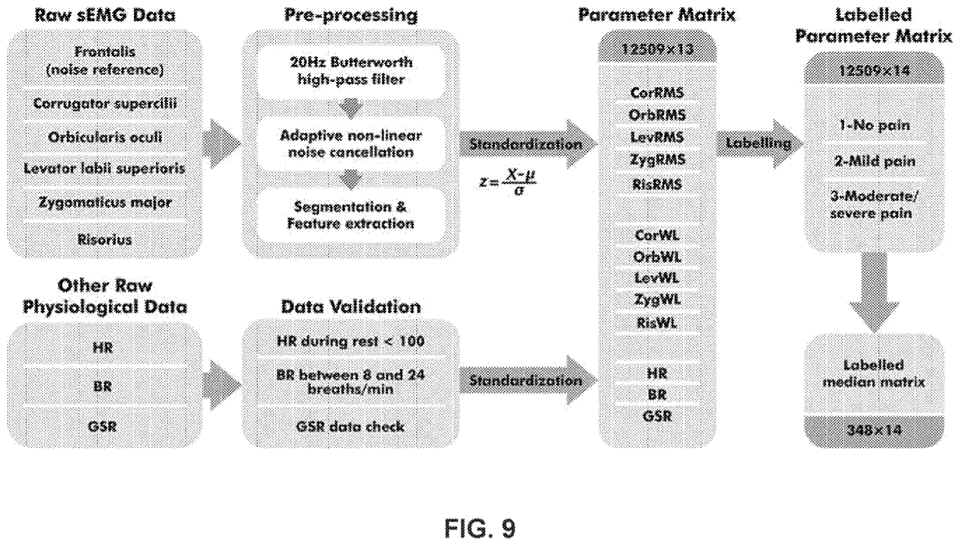

Data on sEMG and other physiological data were processed and checked separately, as shown in FIG. 9. The aim of the pre-processing was to eliminate noise interference and verify the validation of the data. For sEMG, 50 Hz power line noise was coupled to electrode lead wires from the environment. Movement artifacts and baseline drift in low frequencies both caused noise in the sEMG signal. There was also a third noise source, which was caused by electrical stimulus pulses. Electrical pulses were added to finger skin's surface and captured from facial skin's surface as well, due to the electrical conductivity of the human body. In sEMG pre-processing, a 20 Hz Butterworth high-pass filter was first applied to remove movement artifacts and baseline drift from six sEMG channels. Adaptive noise cancellation was employed for the power line and electrical pulse elimination, where non-linear noise in each of the five pain-related facial muscle channels was estimated by reference to a frontalis sEMG with an adaptive neuro-fuzzy inference system (ANFIS) estimator.

To unify the time granularity of sEMG data and other physiological data, sEMG data was split into 1000-sample segments for feature extraction. The root mean square (RMS) in equation (1) and wavelength (WL) in equation (2) were the chosen features, where N was the window length and xi was the i^{th} data point in the window. The RMS feature provided direct insight on sEMG amplitude in order to provide a measure of signal power, while WL was related to both waveform amplitude and frequency [30]. All signal processing was conducted in MATLAB.

For all physiological features, data validation on range and constraint was carried out. After checking, three thermal stimuli tests were excluded from the total of 120 tests due to invalid GSR data in the no pain part, and another thermal stimulus test was excluded for invalid sEMG data. All the validated physiological features were standardized with a standard core within each test and constituted the 13-dimensional parameter matrix. This standardization rescaled the range and distribution of each parameter, in which way the within-subject and between-subject difference in value range was suppressed. There were 12,509 samples at one-second resolution from 116 tests in the parameter matrix. Each sample with 13 parameters was labeled according to the data division in FIG. 2. No pain, mild pain, and moderate/severe pain data were labeled as 1, 2, and 3 respectively. Subsequently, the statistical median of every parameter was calculated from three sections of each test and constituted the median matrix with a length of 348.

### Data Observation and Classification

To visualize the median matrix in 2-dimensional scatter plots, the dimension of parameters in the median matrix was first reduced from 13 with principal component analysis. The first two principal components of the median matrix were non-normally distributed. Nevertheless, with the ability of multivariate analysis, Gaussian distributions were then estimated for each pain intensity level to observe their approximate distribution boundaries in the first two principal components. To fit Gaussians to the parameters of each group, the mean (μ) and variance (σ2) of Gaussian distribution were estimated in maximum likelihood estimation. In a d-dimensional Gaussian distribution, mean and variance were estimated from

$$\begin{matrix}{\hat{μ}}_{i} = \frac{1}{N} {\sum }_{n = 1}^{N} {x}_{ni} , for i = 1 , … d & ( 5 ) \\ {\hat{σ}}_{ij} = \frac{1}{N} {\sum }_{n = 1}^{N} ( {x}_{ni} - {\hat{μ}}_{i} ) ( {x}_{nj} - {\hat{μ}}_{j} ) , for i , j = 1 , … d & ( 6 )\end{matrix}$$

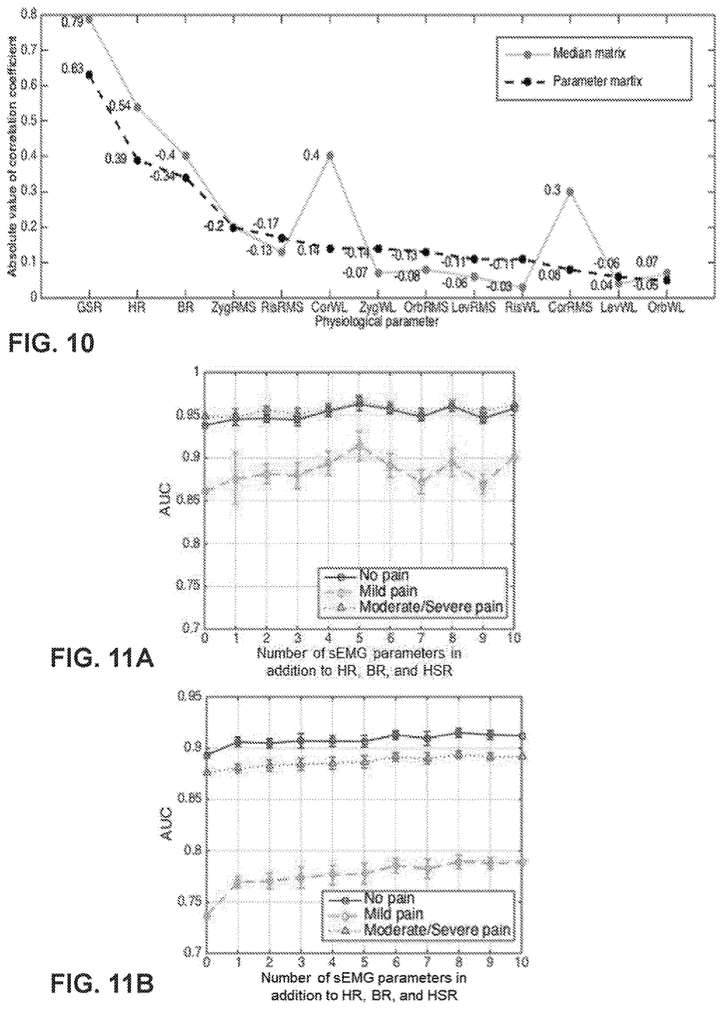

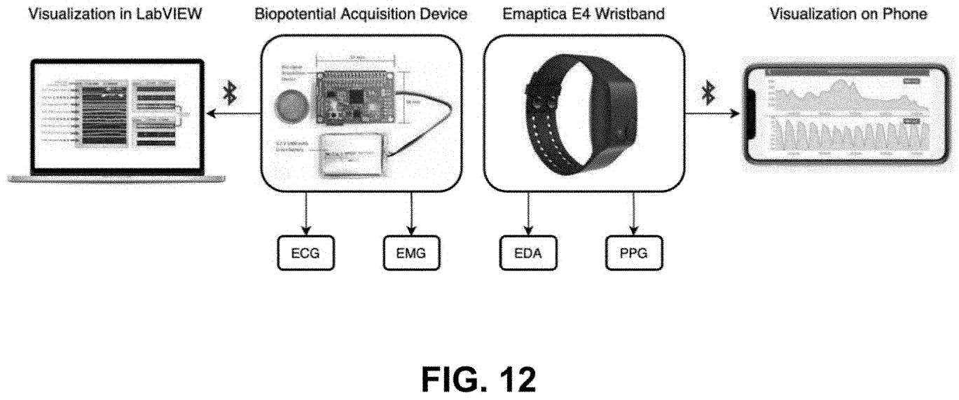

The 95% confidence regions of distributions were marked as approximate boundaries. Tests with different pain stimuli were plotted separately. The significance of each parameter in pain intensity level recognition was observed with correlation analysis. Pearson's linear correlation coefficients between each standardized parameter and labels were calculated, as shown in FIG. 10.

Using the classification method in machine learning, a model can be built to predict class labels (i.e. 1—No pain, 2—Mild pain and 3—Moderate/Severe pain) from input features (i.e. parameter matrix or median matrix). The resulting classifier is then used to assign class labels to the testing instance with new input features. One benefit of applying classification is its effectiveness in establishing many-to-many mapping. The classification technique chosen in this study was artificial neural network (ANN), which is a non-linear classifier having generally better performance with continuous and multi-dimensional features. This method emulates the information processing capabilities of human brain neurons and can provide flexible mapping between inputs and outputs.

In some embodiments, the present invention may implement automatic feature extraction through use of a deep neural network trained with previous data of compressed or latent representations of various signals (e.g. ECG, EDA, PPG). The neural network may be capable of efficiently compressing and encoding data into a lower-dimensional space with minimal reconstruction loss. The number of dimensions of the encoded data corresponds to the number of automatic features extracted. In some embodiments, the number of automatic features extracted by the neural network can be adjusted. Surprisingly, automatic feature extraction as implemented in the present invention is far more efficient for large datasets than human handcrafted features. The size of the dataset is proportional to the number of labels. So by using weak supervision to generate more labels the present invention was able to increase the size of the dataset which helped automatic feature extraction methods.

With 13 parameters as the classifier inputs and 3 pain intensity levels as the outputs, the ANNs classifier was built in three layers: an input layer with 13 units, a hidden layer with 10 units, and an output layer with 3 units. The classifier was applied to both the labeled median matrix and the labeled parameter matrix. Before classification, the samples were divided randomly into three proportions, where 70% were training samples being presented initially to the classifier for training the network; 15% were validation samples to improve classifier generalization properly; and the remaining 15% were testing samples, independent from the trained classifier for classifier performance measurement. The classifier in this work was trained and evaluated in MATLAB Neural Network Toolbox®. The receiver operating characteristic (ROC) curve of each classification was presented. Both average accuracy and the area under ROC curve (AUC) were evaluated as the performance of classification. The true positive rate (TPR) was also taken into consideration in the evaluation, indicating the correct recognition rate of each pain intensity level. The distributions of AUC in classification with different number of involved parameters are shown in FIGS. 11A and 11B.

Thus, patterns of self-reported acute pain intensity levels from monitored physiological signals were observed, which were categorized into no pain, mild pain, and moderate/severe pain based on reported VAS.

### Example 2

The following is another non-limiting example of the present invention. It is to be understood that said example is not intended to limit the present invention in any way. Equivalents or substitutes are within the scope of the present invention.

A biomedical data collection study was conducted on 25 post-operative patients reporting various degrees of pain symptoms. Multimodal biosignals (ECG, EMG, EDA, PPG) were collected from patients likely having mild to moderate pain, who were asked to perform a few light physical activities while acquiring data. All signals were collected using the iHurt system.

iHurt is a system that measures facial muscle activity (i.e., changes in facial expression) in conjunction with physiological signals such as heart rate, heart rate variability, respiratory rate, and electrodermal activity for the purpose of developing an algorithm for pain assessment in hospitalized patients. The system used the two following components to capture raw signals.

Eight-Channel Biopotential Acquisition Device: The team at the University of Turku, Finland developed a biopotential acquisition device to measure ECG and EMG signals. The device incorporated commercially available electrodes, electrode-to-device lead wires, an ADS1299-based portable device, and computer software (LabVIEW version 14.02f, National Instruments) to visualize data streaming from the portable device. Raw signals from the electrodes were sampled at 500 samples per second and were sent to the computer software via Bluetooth for visualization.

Empatica E4: The commercially available Empatica E4 wristband (Empatica Inc, Boston, Mass., USA) was used to measure EDA and PPG signals. The purpose of using a wristband was to allow participants to move freely without any impediments. The Empatica E4 was connected to the participants' phone over Bluetooth for visualization. FIG. 1 presents a visual depiction of the system.

This was the first claimed study that collected biosignals from postoperative adult patients in hospitals. All participants (age: 23-89 years) were recruited from the University of California, Irvine Medical Center after obtaining Institutional Review Board approval (IRB, HS: 2017-3747). 3 participants' data were removed from the final dataset due to the presence of excessive motion artifacts. 2 additional patients were also excluded since they were wearing the Empatica E4 watch on their IV arm, which resulted in unreliable EDA signals due to conditions like skin rash and itching. This left data from 20 patients to build the pain recognition system. The dataset also contained rich annotation with self-reported pain scores based on the 11-point Numeric Rating Scale (NRS) from 0-10.

The first step in building the multimodal pain assessment system was to process the raw signals collected during trials. Data processing pipeline consisted of the following steps: The signal was filtered to remove powerline interference, baseline wander, and motion artifact noise. Feature extraction was performed on the filtered signals to obtain amplitude, time, and frequency domain features. The time-domain features were extracted using 5.5 second windows for the sake of comparison with the state of the art. In addition to handcrafted features, automatic features were used which were outputted from a deep neural network. Once the features were extracted, they were tagged with their corresponding labels based on the nearest timestamp within 5.5 seconds of the label.

Each of these processing steps was applied individually to each of the four modalities. Processed data from each of the modalities were combined using either early fusion or late fusion (explained in detail in the next section). The types of handcrafted features extracted from each modality and the deep learning pipeline for extracting automatic features are described in detail below.

The ECG channel was filtered using a Butterworth band-pass filter with the frequency ranges of [0.1, 250] Hz. ECG handcrafted features (i.e., heart rate variability (HRV)) were extracted from a 5.5 seconds window to make the results comparable to state-of-the-art. The HRV handcrafted features were extracted with pyHRV, an open-source Python toolbox using the R-peaks extracted from ECG signal via a bidirectional long short-term memory network. These features were both time-domain and frequency-domain. There were 19 time-domain (TD) and 13 frequency-domain (FD) extracted features. The TD features extracted from NN intervals, or the time interval between successive R-peaks, comprised of slope of NN intervals, 5 NN interval features (total count, mean, minimum, maximum, and standard deviation), 9 NN interval difference features (mean difference, minimum difference, maximum difference, standard deviation of successive interval differences, root mean square of successive interval differences, number of interval differences greater than 20 ms and 50 ms, and percentage of successive interval differences that differ by more than 20 ms and 50 ms), and 4 heart rate features (mean, minimum, maximum, and standard deviation). The FD features extracted via estimating the power spectral density (PSD) comprised of total power (total spectral power over all frequency bands, 4 High Frequency (HF) band fast fourier transform (FFT) features (peak, absolute, relative, and normalized), 3 very low frequency (VLF) band FFT features (peak, absolute, relative), and FFT ratio of HF and low frequency (LF) bands. This resulted in 32 features in total.

The preprocessing phase of EMG channels comprised of a 20 Hz Highpass filter and two notch filters at 50 Hz and 100 Hz all using a Butterworth filter. Similar to ECG features, EMG features were extracted from a 5.5 second window. However, features from various domains including amplitude, frequency, entropy, and variability were also extracted. The 10 amplitude features extracted were 1) peak, 2) peak to peak mean value (p2pmv), 3) root mean squared (rms), 4) mean of the absolute values of the second differences (mavsd), 5) mean of the absolute values of the first differences (mavfd), 6) mean of the absolute values of the second differences of the normalized signal (mavsdn), 7) mean of the absolute values of the first differences of the normalized signal (mavfdn), 8) mean of local minima values (mlocminv), 9) mean of local maxima values (mlocmaxv), and 10) mean of absolute values (may). The 4 frequency features extracted were 1) Median Frequency, 2) bandwidth frequency at 3 dB, 3) center frequency, and 4) mode freq. The 3 Entropy features were 1) Approximate Entropy, 2) Sample Entropy, and 3) Spectral Entropy. The 4 Variability features were 1) Variance, 2) Standard deviation, 3) Range, and 4) Interquartile Range. All 21 aforementioned features were calculated for 5 different EMG channels resulting in 105 EMG features.

The pyEDA library was used for pre-processing and feature extraction of EDA signals. In the pre-processing part, first, a moving average was used across a 1-second window to remove the motion artifacts and smooth the data. Second, a low-pass Butterworth filter on the phasic data was applied to remove the line noise. Lastly, preprocessed EDA signals corresponding to each different pain level were visualized to ensure the validity of the signals. In the feature extraction part, cvxEDA algorithm was employed to extract the phasic component of EDA signals. The EDA signals' peaks or bursts were considered the variations in the phasic component of the signal. Therefore, the clean signals and extracted phasic component of signals were fed to the statistical feature extraction module to extract the number of peaks, the average value, and the maximum and minimum value of the signals. Moreover, these extracted features were further employed in the post-feature extraction module to extract 8 more features: (1) The difference between the maximum and the minimum value of the signal, (2) Standard deviation of the signal, (3) The difference between upper and lower quartiles of the signal, (4) Root mean square of the signal, (5) The mean value of local minima of the signal, (6) The mean value of local maxima of the signal, (7) The mean of the absolute values of the first differences, and (8) The mean of the absolute values of the second differences. This resulted in 12 EDA features in total.

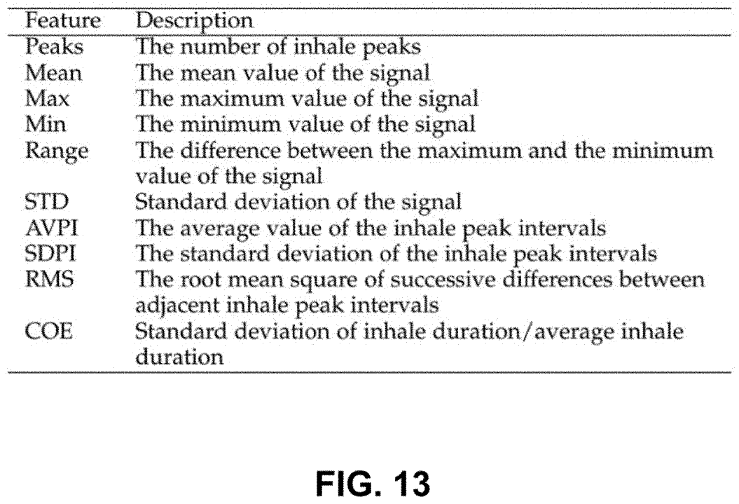

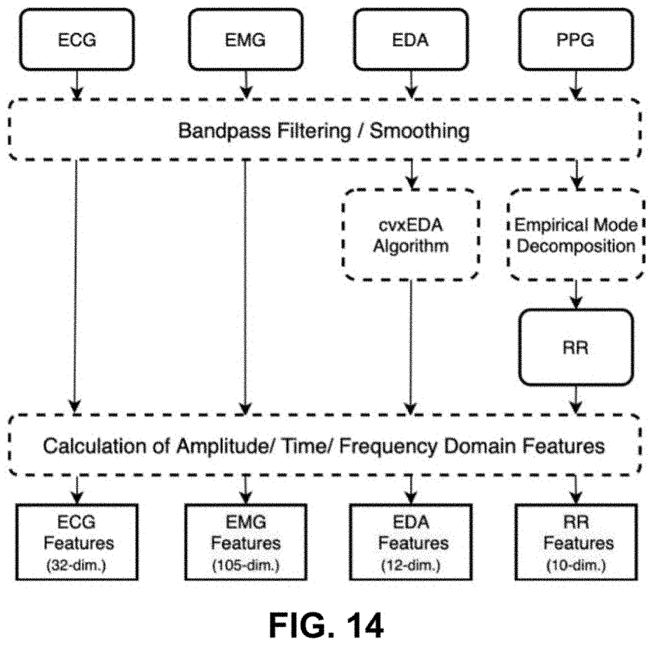

The PPG signal was pre-processed before extracting the respiratory rate from it. Two filters were used during the preprocessing. A Butterworth bandpass filter was first used to remove noises including motion artifacts. Then, a moving average filter was implemented to smooth the PPG signal. After that, an Empirical Mode Decomposition (EMD) based method was applied to derive respiration signals from filtered PPG signals. This method was proved to derive RR from a PPG signal with high accuracy (99.87%). Ten features were extracted from the respiratory signal and are briefly described in FIG. 13. A visualization of the handcrafted feature pipeline is shown in FIG. 14.

As the dimensionality of biomedical data increases, it becomes increasingly difficult to train a machine learning algorithm on the entire uncompressed dataset. This often leads to a large training time and was computationally more expensive overall. One possible solution was to perform feature engineering to get a compressed and interpretable representation of the signal. Another alternative approach, however, was to use the compressed or latent representation of that data obtained from deep learning networks trained for that specific task. Using automatic features helps in dimensionality reduction and can provide us with a sophisticated yet succinct representation of the data that handcrafted features alone cannot provide. This automatic feature extraction was typically carried out by an autoencoder network, which is an unsupervised neural network that learns how to efficiently compress and encode the data into a lower-dimensional space. Autoencoders are composed of two separate networks, a decoder, and an encoder. The decoder network acts as a bottleneck layer and maps the input into a lower-dimensional feature space. The encoder network tries to reconstruct this lower-dimensional feature vector into the original input size. The entire network was trained to minimize the reconstruction loss (i.e mean-squared error) by iteratively updating its weights and biases through backpropagation.

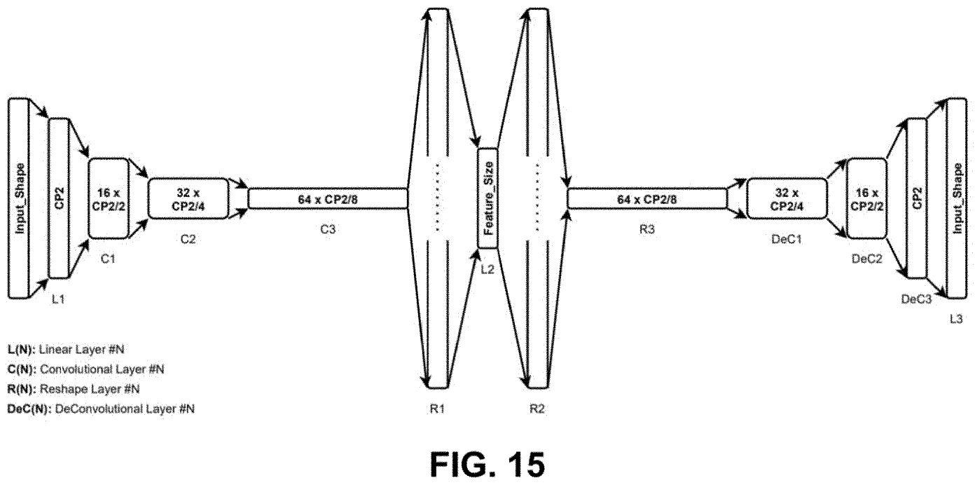

A convolutional autoencoder from the pyEDA library was used to extract automatic features. FIG. 15 shows the architecture of the autoencoder. First, a linear layer (L1) was used to downsample the input signal with Input_Shape length to a length that was the closest power of 2 (CP2). This was done to make the model scalable to an arbitrary input size. The encoder half of the network consists of three 1-D convolutional layers (C1, C2, and C3) and a linear layer (L2) which flatten and downsamples the input vector to a lower-dimensional latent vector. The number of dimensions of this latent vector (Feature_Size) corresponds to the number of automatic features extracted and was set prior to training the network. A total of 32 features were extracted from ECG, EDA, and RR signals. Whereas, a total of 30 features were extracted from the EMG signal (6 features from each of the 5 channels). The decoder half of the network consists of three 1-D de-convolutional layers (DeC1, DeC2, and DeC3) to reconstruct the input signal from the latent vector. A final linear layer (L3) was then used to flatten and reconstruct the signal to its original dimension. Both encoder and decoder networks have ReLU (Rectified Linear Unit) activation between layers. A window size of 10 seconds was applied to the filtered signals to provide the model with more temporal context. Furthermore, the input vector length using 10-second windows (as opposed to 5.5-second windows) seemed to generalize better among different sampling rates across modalities. After signals from each of the modalities were normalized, they were divided into 10-second chunks and trained on separate autoencoder models.

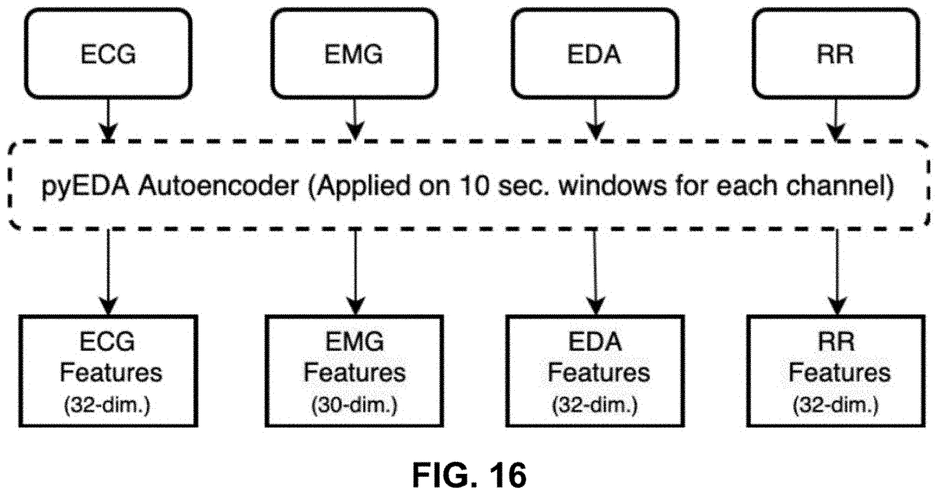

The batch size was set to 10, the number of training epochs was set to 100, and the ADAM optimizer was used with a learning rate of 1e−3. A total of 126 features across all 4 modalities were extracted. A visualization of the automatic feature extraction pipeline is shown in FIG. 16.

There were a number of inherent challenges in the distribution of labels as NRS values recorded during the clinical trials of this study were collected from real postoperative patients. This problem bears less significance while studying healthy participants since the stimulated pain can be controlled during the experiments. As a consequence, occurrences of some pain levels far exceeded those of others. For example, among all patients, there were only 4 reported occurrences of pain level 10, whereas there were more than 80 reported occurrences of pain level 4. This imbalanced distribution was inevitable due to the subjective nature and the different sources of pain among the participants. Therefore, while downsampling the pain labels to 5 classes, thresholds for each downsampled class were carefully chosen to ensure a more evenly distributed set of labels. Moreover, since the NRS values were only reported after performing some pain-stimulating activities, labels were stored sparsely. The handcrafted features were combined with the corresponding labels using timestamps that were within the nearest 5.5 seconds (labeling threshold) of the reported NRS value. The automatic features used a labeling threshold of 10 seconds instead. As a consequence of having sparse labels, many of the feature windows were not assigned a corresponding label. To mitigate the problem of having an imbalanced and sparse label distribution, two techniques were exploited.

The first technique, called Synthetic Minority Oversampling (Smote), is a type of data augmentation that over-samples the minority class. Smote works by first choosing a minority class instance at random and finding its k nearest minority class neighbors. It then creates a synthetic example at a randomly selected point between two instances of the minority class in that feature space. The experiments involving Smote were implemented using the imbalanced-learn python library.

The second technique utilized was weak supervision using the Snorkel general-purpose framework. Rather than employing an expert to manually label the unlabelled instances, Snorkel allows its users to write labeling functions that can make use of heuristics, patterns, external knowledge bases, and third-party machine learning models. It is an application-independent platform and can be used for any type of data ranging from healthcare to self-driving cars. Weak supervision is used to describe machine learning algorithms that implement indications and imprecise/unorganized data to label a large amount of data. Labelling this data allows it to be used in other machine learning algorithms whereas the original unorganized data could not be used.

Weak supervision was typically employed to label large volumes of unlabeled data when there were noisy, limited, or imprecise sources. For the purpose of the pain assessment algorithm, third-party machine learning models were used to label the remaining unlabelled instances. All the data points that were within the labeling threshold were considered as “strong labels”, or ground-truth values collected from patients during trials. The remaining unlabelled data points were kept aside for Snorkel to provide a weakly supervised label. The strong labels were fed into Snorkel's labeling function consisting of three off-the-shelf machine learning models: (i) a Support-Vector Machine (SVM) with a radial basis function kernel, (ii) a Random Forest (RF) classifier, and (iii) a K-Nearest Neighbor (KNN) classifier with uniform weights. Once each model was trained on the strong labels, it was used to make predictions on the remaining unlabeled data. The predictions from these three models were collected and converted into a single confidence-weighted label per data point using Snorkel's “LabelModel” function. This function outputs the most confident prediction as the label for each data point. To perform a fair assessment of the reliability and accuracy of the algorithm, Smote and Snorkel only were used while training the machine learning models. The performance of these models was measured solely on ground-truth (strong) labels collected during trials. This way, there was no implicit bias introduced from mislabeling or up-sampling certain data points to skew model predictions.

To compare the performance of the multimodal machine learning models with the prior work, binary classification was performed using a leave-one-subject-out cross-validation approach. In this method, a model's performance was validated over multiple folds in such a way that data from each patient was either in the training set or in the testing set. The purpose of using this method was to provide generalizability to unseen patients and to avoid overfitting by averaging the results over multiple folds. The eventual goal of this study was to build personalized models that make predictions on a single patient but learn from data collected from a larger population of similar patients. The following machine learning models were used to evaluate the performance of the pain assessment algorithm: (1) k-nearest neighbor with k ranging from 1 to 50, (2) random forest classifier with a depth ranging from 10 to 100, (3) AdaBoost (Adaptive Boosting) with the number of base estimators ranging from 20 to 2000, (4) and a SVM (Support Vector Machine) with a radial basis function kernel and a degree of 3. The optimal hyperparameter settings for these models were obtained using randomized grid search with 3-fold cross-validation. The best parameters were selected for each model and they were then evaluated using leave-one-subject-out cross-validation. Four separate models were trained for each of the four pain intensities (e.g BL, no pain versus PL1, the lowest pain level, or BL vs PL4, the highest pain level).

Two fusion approaches were used while combining features across different modalities. The first one is early or feature-level fusion which concatenates feature vectors across different modalities based on their timestamps. The resulting data that was higher in dimension than any one single modality was then fed into the classifier to make predictions. While concatenating features across different modalities, a threshold of 5.5 seconds was used to combine all hand-crafted features and a threshold of 10 seconds was used to combine the automatic features. There were a total of 159 and 126 different features amongst the handcrafted and automatic features, respectively. The second approach was late or decision level fusion where each modality was fed to a separate classifier and the final classification result was based on the fusion of outputs from the different modalities.

Since there were a lot of features generated during the data processing phase, a subset of the most informative features had to be selected to build the models with. Therefore, to reduce the complexity and training time of the resulting model, feature selection using Gini importance was performed. To obtain the best set of features for the classification models, a leave-one-person-out cross-validation fold was carried out on the four different models for each pain intensity using an AdaBoost classifier. The Gini importance of the features was computed from the training data and selected the top n features (where n ranged from 10 to 50 in increments of 10). Since there were multiple folds, it was possible to have different sets of features for each of the folds. The most commonly selected features across all folds were considered as the final set of features to use for the model. In this way, each of the pain intensity models could have a different subset of features across each of the four modalities because these models operate independently of each other.

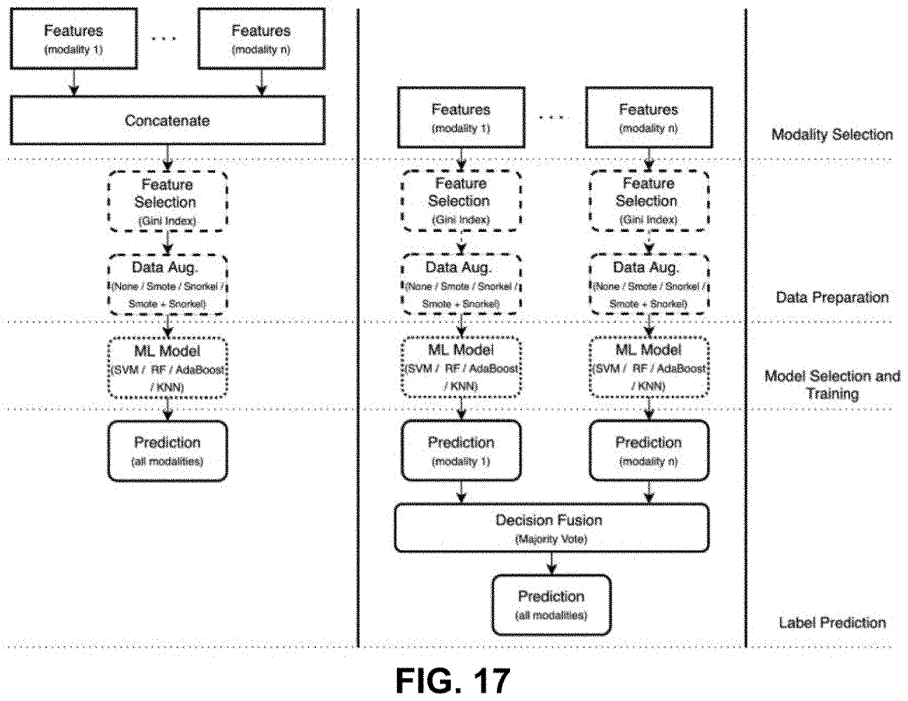

The goal of these experiments was to compare the performance of using only a single modality to build the models over using a combination of multiple modalities. Several different models were trained for each of the pain intensities that varied in the types of modalities, data augmentation techniques, machine learning models, and fusion techniques used. FIG. 17 shows the general pipeline of the experiments conducted. First, the type of modalities to train on was selected, which varied from only using each of the single modalities separately to using a combination of 3 or more modalities. Moreover, these modalities varied on the types of features used, like handcrafted or automatic features. In the case of using multiple modalities, there were two choices of fusion; early (FIG. 17 left) and late (FIG. 17 right). These architectures varied in how the modalities were combined, either before training (early), or at decision level (late) after training using majority voting. The data preparation process involved feature selection and data augmentation. These models could either be trained with no data augmentation, with just Smote or Snorkel, or a combination of both of them. The last step of the pipeline before making predictions involved choosing the type of machine learning algorithms, like SVM, Random Forest (RF), Adaptive Boosting (AdaBoost), or K-Nearest Neighbors (KNN). Due to the lack of space, only the best-performing single and multimodal model configurations are mentioned in the section below.

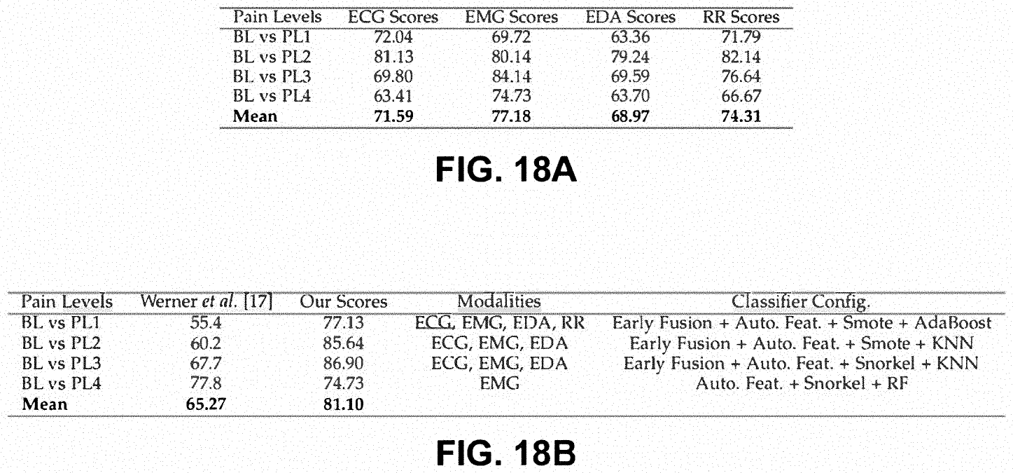

FIGS. 18A-18B present the best performing single modal and multimodal models for each of the four pain intensities. The best multimodal models are mentioned along with their ML algorithm, data augmentation, and feature engineering methods employed to achieve their results. For comparison, the best multimodal results from prior experiments are also mentioned.

From the single modality results (FIG. 18A), it was evident that EMG models outperform all other modalities especially for the BL vs PL3 and BL vs PL4 models. Overall, models from all modalities have relatively lower scores in the BL vs PL1 and BL vs PL4 pain groups. The comparatively lower performance of EDA models over other modalities suggests that variations in EDA signal response to different pain levels were more difficult to distinguish. Moreover, the EDA signals were collected using the Empatica E4 wrist-worn device which makes them more prone to motion artifacts during trials. The multimodal results (FIG. 18B) suggest that the best-performing models were built using automatic features. Most of these models used either one of the data augmentation techniques or a combination of both. The improved results obtained using such data augmentation like Smote and Snorkel were understandable due to the imbalanced distribution and sparse nature of labels collected from post-operative patients.

The relatively poor performances of the BL vs PL1 and BL vs PL4 models across both single and multimodal models were also understandable because they lie at the extremes of the pain threshold. The BL vs PL1 models might find it more challenging to distinguish between baseline levels and the lowest pain intensity due to the subtlety of the physiological responses collected while experiencing this pain level. The BL vs PL4, however, might find it challenging to distinguish pain levels due to the scarcity of such labels collected during trials. Data augmentation can help mitigate this problem, but there was no substitute for real data. Moreover, most single modality models for the highest pain intensity trained without any data augmentation techniques only performed as well as random guessing (˜50%) and sometimes even worse (not shown in the table). On the contrary, the models for the middle two pain intensities performed better due to the relative abundance of such labels reported during trials. It should be noted that accuracy was used as a validation metric instead of F1 and AUC scores because a lot of the patients did not experience all of the pain levels (BL, . . . , PL4). As a consequence, their true and false-positive rates were not computed.

In terms of modalities, the best-performing models used EMG either alone or in combination with other signals. One justification for this could be due to the dynamic nature of EMG signals collected from facial muscles while experiencing pain. Since periods of higher pain intensity were effectively isolated and captured with smaller window sizes, this helped the models better distinguish between baseline and other pain levels. This was especially evident in the BL vs PL4 models, where EMG alone provided the best results for both single modal and multimodal models.

The best-performing multimodal models used a combination of early fusion or feature level fusion along with a data augmentation technique. One intuition as to why early fusion might have performed better overall was due to the detection of correlated features across modalities obtained after using feature selection. Late fusion, on the contrary, built independent models for each modality and fuses them based on their predictions using majority voting. Therefore, by treating each modality as independent, there was a potential loss of correlation in the combined feature space.

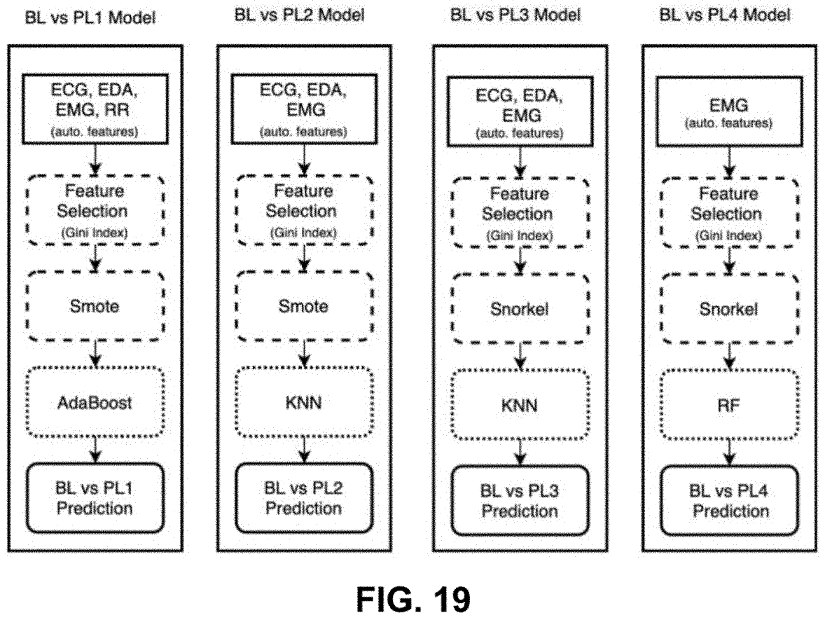

Overall, the multimodal models outperform all the single modal models in the first three pain intensities. It was clear that using multiple modalities enhances the models' ability to distinguish between different pain levels. The single modality results, however, can provide us with some key insights on which modality to prioritize in the absence of other modalities. A visualization of the best-performing models is shown in FIG. 19. While comparing these results to prior systems, the models of the present invention outperform their models in all pain classes except the last one. This was understandable because the data was not collected from healthy subjects in controlled environments where the label distributions were more balanced.

The present invention features a multimodal machine learning framework for classifying pain in real post-operative patients from the iHurt Pain Database. Both traditional handcrafted features and deep learning generated automatic features were extracted from physiological signals (ECG, EDA, EMG, PPG). Several experiments were conducted to perform binary classification among four different pain intensities vs baseline levels of pain. Models for each of these intensities were varied based on the modalities used, the different types of data augmentation techniques (Smote, Snorkel, or both), the machine learning algorithms used, and the type of modality fusion used. These results showed that binary pain classification greatly benefits from using data augmentation techniques in conjunction with automatic features. The multimodal model outperformed the single modal models, with the exception of the last pain intensity. The BL vs PL4 model with the best results was trained on EMG data alone, which suggests that facial muscle activation can play a vital role in distinguishing higher pain intensities from baseline levels of pain. This was consistent from a clinical perspective because higher pain intensities were more commonly associated with acute pain.

However, since pain is a subjective experience that tends to have a large inter-individual variability, building a monolithic model for all patients might not be a viable solution. A promising future direction for this research study was to build personalized machine learning models that can benefit from using data from groups of similar patients, but which were fine-tuned to make predictions on a single person. Prior research has used multitask machine learning (MTL) to account for inter-individual variability and build personalized models for the task of mood prediction. This was a feasible future research direction that would be applicable to the domain of pain assessment, not only for the acute pain of surgery but also for patients that experience chronic pain. It was believed that personalized modeling will be a vital step in creating clinically viable pain assessment algorithms.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for integrating surface electromyogram (sEMG) signals and physiological signals for automatically detecting pain intensity levels experienced by a human without use of a camera, wherein the method comprises:
(a) providing a wearable facial expression capturing system (100) for measuring said pain intensity levels, the system (100) comprising:
(i) a flexible mask (102) contoured to at least partially cover one side of the human's face (114), the mask having an eye recess or opening (115) disposed between an elongated forehead portion (116) of the mask, which is above the eye recess (115), and a cheek portion (117) of the mask, which is beneath the eye recess (115);
(ii) at least two electrodes (104) disposed in the mask (102), wherein a first electrode is disposed in the forehead portion of the mask (116), and a second electrode is disposed in the cheek portion of the mask (117);
(iii) a sensor node (108) disposed on a lateral flap (118) extending from the cheek portion of the mask, wherein the sensor node (108) comprises a processing module and a transmitter (112); and
(iv) connecting leads (106) electrically coupling each of the at least two sensors (104) to the sensor node (108);
(b) applying the flexible mask to partially cover one side of the human's face such that the first electrode aligns with a corrugator facial muscle and the second electrode aligns with a zygomatic facial muscle;
(c) detecting sEMG signals from the corrugator facial muscle and the zygomatic facial muscle via the first and second sensors, respectively;
(d) filtering the detected sEMG signals via the processing module;
(e) transmitting the filtered sEMG signals to a data fusion system (322) via the wireless transmitter (308);
(f) transmitting physiological signals from one or more wearable sensors (312) to the data fusion system (322), the physiological signals comprising one or more of a breath rate, a heart rate, a galvanic skin response (GSR), a skin temperature signal, or a photoplethysmogram (PPG) signal;
(g) processing, by the data fusion system (322), the sEMG signals and the physiological signals to determine the pain intensity levels, comprising:
i. labelling, by a weak supervision algorithm, the sEMG signals and the physiological signals;
ii. extracting features from each of the sEMG signals and the physiological signals;
iii. performing feature alignment on features extracted from the sEMG signals and the physiological signals;
iv. performing interindividual standardization on each of the sEMG signals and the physiological signals;
v. performing pattern recognition by comparing the sEMG signals and the physiological signals to a database;
vi. correlating patterns recognized with pain intensity levels and classifying the pain intensity levels; and
(h) displaying the pain intensity levels to a medical care provider, thus allowing for continuous and automatic pain monitoring.

2. The method of claim 1, wherein extracting features from each of the sEMG signals and the physiological signals comprises a root-mean-square (RMS) feature extraction and a wavelength (WL) feature extraction.

3. The method of claim 1, wherein performing feature alignment includes synchronizing the sEMG signals and the physiological signals by using cross-correlation functions.

4. The method of claim 1, wherein a multimodal artificial neural network classifier correlates patterns recognized with pain intensity levels and classifies the pain intensity levels.

5. The method of claim 1, wherein the flexible mask (102) is composed of polydimethyl silicone elastomer (PDMS).

6. The method of claim 1, wherein the sensors (104) comprise Ag/AgCl electrodes.

7. The method of claim 6, wherein the electrodes are formed on an inner surface of the mask (102) such that the electrodes are directly contacting skin when the mask is applied to the human's face.

8. A method for integrating surface electromyogram (sEMG) signals and physiological signals for automatically detecting pain intensity levels experienced by a human without use of a camera, wherein the method comprises:
(a) receiving the sEMG signals from a wearable facial expression capturing system (302) placed on the human's face (114), wherein said system (302) comprises (i) a mask embedded with a plurality of sensors (304) at locations that line up with specific facial muscles, wherein the sensors are configured to detect sEMG signals from the facial muscles; (ii) a sensor node (306) configured to analyse the sEMG signals detected by the electrodes; (iii) a processing module (310) configured to filter the sEMG signals; and (iv) a wireless transmitter (308) configured to wirelessly transmit the filtered sEMG signals to a data fusion system (322);
(b) transmitting physiological signals from one or more wearable sensors (312) to the data fusion system (322), the physiological signals comprising one or more of a breath rate, a heart rate, a galvanic skin response (GSR), or a photoplethysmogram (PPG) signal;
(c) processing, by the data fusion system (322), the sEMG signals and the physiological signals to determine the pain intensity levels, comprising:
i. performing feature alignment on features extracted from the sEMG signals and the physiological signals;
ii. extracting features from each of the sEMG signals and the physiological signals;
iii. performing interindividual standardization on each of the sEMG signals and the physiological signals;
iv. performing pattern recognition by comparing the sEMG signals and the physiological signals to a database;
v. correlating patterns recognized with pain intensity levels and classifying the pain intensity levels; and
(d) displaying the pain intensity levels to a medical care provider, thus allowing for continuous and automatic pain monitoring.

9. The method of claim 8, wherein extracting features from each of the sEMG signals and the physiological signals comprises a root-mean-square (RMS) feature extraction and a wavelength (WL) feature extraction.

10. The method of claim 8, wherein performing feature alignment includes synchronizing the sEMG signals and the physiological signals by using cross-correlation functions.

11. The method of claim 8, wherein correlating patterns recognized with pain intensity levels and classifying the pain intensity levels is done by an artificial neural network classifier.

12. A facial expression capturing system (100) for measuring pain levels experienced by a human without use of a camera, the system (100) comprising:
a) a flexible mask (102) contoured to at least partially cover one side of the human's face (114), the mask having an eye recess or opening (115) disposed between an elongated forehead portion (116) of the mask, which is above the eye recess (115), and a cheek portion (117) of the mask, which is beneath the eye recess (115);
b) six sensor positions located on the mask (102) such that two sensor positions are located laterally on the elongated forehead portion (116) of the mask and the other four sensor positions located on the cheek portion (117) of the mask and situated in a 2 by 2 arrangement;
c) two or more sensors (104) embedded in the mask (102), wherein each sensor occupies one of the sensor positions;
d) a sensor node (108) disposed on a lateral flap (118) extending from the cheek portion (117) of the mask, wherein the sensor node (108) comprises a processing module and a transmitter (112); and
e) connecting leads (106) electrically coupling each of the two or more sensors (104) to the sensor node (108);
wherein when the flexible mask is applied to partially cover one side of the human's face, the sensor positions align with pain-related facial muscles in the human's face, wherein the sensors (104) are configured to detect biosignals from underlying facial muscles, wherein the processing module is configured to: (i) receive the biosignals from the plurality of sensors, (ii) analyze the biosignals to deduce facial expressions and monitor pain intensity levels experienced by the subject based on the deduced facial expressions, and (iii) transmit the pain intensity levels to a medical care provider, thus allowing the medical care provider to continually monitor the pain intensity levels experienced by the subject thereby providing effective and efficient pain management.

13. The system (100) of claim 12, wherein the flexible mask (102) is composed of polydimethyl silicone elastomer (PDMS).

14. The system (100) of claim 12, wherein the sensors (104) comprise Ag/AgCl electrodes.

15. The system (100) of claim 14, wherein the electrodes are formed on an inner surface of the mask (102) such that the electrodes are directly contacting skin when the mask is placed on the human's face.

16. The system (100) of claim 12, wherein the pain-related facial muscles are frontalis, corrugator, orbicularis oculi, levator, zygomaticus, and risorius.

17. The system (100) of claim 12 comprising two sensors (104), wherein a first sensor occupies a distal-most sensor position located on the forehead portion (116) of the mask, wherein a second sensor occupies a first row and first column of the 2 by 2 arrangement in the cheek portion (117) of the mask, wherein the first sensor detects biosignals from a corrugator facial muscle and the second sensor detects biosignals from a zygomatic facial muscle.

18. The system (100) of claim 12 comprising five sensors (104), wherein a first sensor and a second sensor occupy the two sensor positions on the forehead portion (116) of the mask, wherein a third sensor and a fourth sensor occupy the sensor positions at a first row of the 2 by 2 arrangement in the cheek portion (117) of the mask, wherein a fifth sensor occupies the sensor position at a second row and second column of the 2 by 2 arrangement, wherein the first sensor detects biosignals from a corrugator facial muscle, the second sensor detects biosignals from a frontalis facial muscle, the third sensor detects biosignals from a levator facial muscle, the fourth sensor detects biosignals from an orbicularis oculi facial muscle, and the fifth sensor detects biosignals from a zygomatic facial muscle.

19. The system (100) of claim 12, wherein the facial expressions comprise one or more of a smile, frown, and a wrinkled nose.

20. The system (100) of claim 12, wherein the biosignals comprise surface electromyogram (sEMG) signals.
